SEQUENCE LISTING (66 sequences)

SEQ ID NO: 1 - DNA, 33 bp, Homo sapiens
aaaaaccgcggtcaattatttctcatcactccc

SEQ ID NO: 2 - DNA, 37 bp, Homo sapiens
aaaaactcgagaaaagaaaagtgtatctctcagagtg

SEQ ID NO: 3 - DNA, 49 bp, Homo sapiens
aaaaactcgagaaaagagaggctgaagctaaagtgtatctctcagagtg

SEQ ID NO: 4 - DNA, 32 bp, Homo sapiens
aaaaattcgaaaaatggaacataaggaagtgg

SEQ ID NO: 5 - DNA, 35 bp, Homo sapiens
aaaaactcgagaaaagagagcctctggatgactat

SEQ ID NO: 6 - DNA, 47 bp, Homo sapiens
aaaaactcgagaaaagagaggctgaagctgagcctctggatgactat

SEQ ID NO: 7 - DNA, 2400 bp, Homo sapiens
atgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagaaaagtgtatctctcagagtgcaagactgggaatggaaagaactacagagggacgatgtccaaaacaaaaaatggcatcacctgtcaaaaatggagttccacttctccccacagacctagattctcacctgctacacacccctcagagggactggaggagaactactgcaggaatccagacaacgatccgcaggggccctggtgctatactactgatccagaaaagagatatgactactgcgacattcttgagtgtgaagaggaatgtatgcattgcagtggagaaaactatgacggcaaaatttccaagaccatgtctggactggaatgccaggcctgggactctcagagcccacacgctcatggatacattccttccaaatttccaaacaagaacctgaagaagaattactgtcgtaaccccgatagggagctgcggccttggtgtttcaccaccgaccccaacaagcgctgggaactttgcgacatcccccgctgcacaacacctccaccatcttctggtcccacctaccagtgtctgaagggaacaggtgaaaactatcgcgggaatgtggctgttaccgtttccgggcacacctgtcagcactggagtgcacagacccctcacacacataacaggacaccagaaaacttcccctgcaaaaatttggatgaaaactactgccgcaatcctgacggaaaaagggccccatggtgccatacaaccaacagccaagtgcggtgggagtactgtaagataccgtcctgtgactcctccccagtatccacggaacaattggctcccacagcaccacctgagctaacccctgtggtccaggactgctaccatggtgatggacagagctaccgaggcacatcctccaccaccaccacaggaaagaagtgtcagtcttggtcatctatgacaccacaccggcaccagaagaccccagaaaactacccaaatgctggcctgacaatgaactactgcaggaatccagatgccgataaaggcccctggtgttttaccacagaccccagcgtcaggtgggagtactgcaacctgaaaaaatgctcaggaacagaagcgagtgttgtagcacctccgcctgttgtcctgcttccagatgtagagactccttccgaagaagactgtatgtttgggaatgggaaaggataccgaggcaagagggcgaccactgttactgggacgccatgccaggactgggctgcccaggagccccatagacacagcattttcactccagagacaaatccacgggcgggtctggaaaaaaattactgccgtaaccctgatggtgatgtaggtggtccctggtgctacacgacaaatccaagaaaactttacgactactgtgatgtccctcagtgtgcggccccttcatttgattgtgggaagcctcaagtggagccgaagaaatgtcctggaagggttgtgggggggtgtgtggcccacccacattcctggccctggcaagtcagtcttagaacaaggtttggaatgcacttctgtggaggcaccttgatatccccagagtgggtgttgactgctgcccactgcttggagaagtccccaaggccttcatcctacaaggtcatcctgggtgcacaccaagaagtgaatctcgaaccgcatgttcaggaaatagaagtgtctaggctgttcttggagcccacacgaaaagatattgccttgctaaagctaagcagtcctgccgtcatcactgacaaagtaatcccagcttgtctgccatccccaaattatgtggtcgctgaccggaccgaatgtttcatcactggctggggagaaacccaaggtacttttggagctggccttctcaaggaagcccagctccctgtgattgagaataaagtgtgcaatcgctatgagtttctgaatggaagagtccaatccaccgaactctgtgctgggcatttggccggaggcactgacagttgccagggtgacagtggaggtcctctggtttgcttcgagaaggacaaatacattttacaaggagtcacttcttggggtcttggctgtgcacgccccaataagcctggtgtctatgttcgtgtttcaaggtttgttacttggattgagggagtgatgagaaataattga

SEQ ID NO: 8 - protein, 799 aa, Homo sapiens
MRFPSIFTAVLFAASSALAAPVNTTTEDETAQIPAEAVIGYSDLEGDFDVAVLPFSNSTNNGLLFINTTIASIAAKEEGVSLEKRKVYLSECKTGNGKNYRGTMSKTKNGITCQKWSSTSPHRPRFSPATHPSEGLEENYCRNPDNDPQGPWCYTTDPEKRYDYCDILECEEECMHCSGENYDGKISKTMSGLECQAWDSQSPHAHGYIPSKFPNKNLKKNYCRNPDRELRPWCFTTDPNKRWELCDIPRCTTPPPSSGPTYQCLKGTGENYRGNVAVTVSGHTCQHWSAQTPHTHNRTPENFPCKNLDENYCRNPDGKRAPWCHTTNSQVRWEYCKIPSCDSSPVSTEQLAPTAPPELTPVVQDCYHGDGQSYRGTSSTTTTGKKCQSWSSMTPHRHQKTPENYPNAGLTMNYCRNPDADKGPWCFTTDPSVRWEYCNLKKCSGTEASVVAPPPVVLLPDVETPSEEDCMFGNGKGYRGKRATTVTGTPCQDWAAQEPHRHSIFTPETNPRAGLEKNYCRNPDGDVGGPWCYTTNPRKLYDYCDVPQCAAPSFDCGKPQVEPKKCPGRVVGGCVAHPHSWPWQVSLRTRFGMHFCGGTLISPEWVLTAAHCLEKSPRPSSYKVILGAHQEVNLEPHVQEIEVSRLFLEPTRKDIALLKLSSPAVITDKVIPACLPSPNYVVADRTECFITGWGETQGTFGAGLLKEAQLPVIENKVCNRYEFLNGRVQSTELCAGHLAGGTDSCQGDSGGPLVCFEKDKYILQGVTSWGLGCARPNKPGVYVRVSRFVTWIEGVMRNN

SEQ ID NO: 9 - DNA, 2412 bp, Homo sapiens
atgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagctaaagtgtatctctcagagtgcaagactgggaatggaaagaactacagagggacgatgtccaaaacaaaaaatggcatcacctgtcaaaaatggagttccacttctccccacagacctagattctcacctgctacacacccctcagagggactggaggagaactactgcaggaatccagacaacgatccgcaggggccctggtgctatactactgatccagaaaagagatatgactactgcgacattcttgagtgtgaagaggaatgtatgcattgcagtggagaaaactatgacggcaaaatttccaagaccatgtctggactggaatgccaggcctgggactctcagagcccacacgctcatggatacattccttccaaatttccaaacaagaacctgaagaagaattactgtcgtaaccccgatagggagctgcggccttggtgtttcaccaccgaccccaacaagcgctgggaactttgcgacatcccccgctgcacaacacctccaccatcttctggtcccacctaccagtgtctgaagggaacaggtgaaaactatcgcgggaatgtggctgttaccgtttccgggcacacctgtcagcactggagtgcacagacccctcacacacataacaggacaccagaaaacttcccctgcaaaaatttggatgaaaactactgccgcaatcctgacggaaaaagggccccatggtgccatacaaccaacagccaagtgcggtgggagtactgtaagataccgtcctgtgactcctccccagtatccacggaacaattggctcccacagcaccacctgagctaacccctgtggtccaggactgctaccatggtgatggacagagctaccgaggcacatcctccaccaccaccacaggaaagaagtgtcagtcttggtcatctatgacaccacaccggcaccagaagaccccagaaaactacccaaatgctggcctgacaatgaactactgcaggaatccagatgccgataaaggcccctggtgttttaccacagaccccagcgtcaggtgggagtactgcaacctgaaaaaatgctcaggaacagaagcgagtgttgtagcacctccgcctgttgtcctgcttccagatgtagagactccttccgaagaagactgtatgtttgggaatgggaaaggataccgaggcaagagggcgaccactgttactgggacgccatgccaggactgggctgcccaggagccccatagacacagcattttcactccagagacaaatccacgggcgggtctggaaaaaaattactgccgtaaccctgatggtgatgtaggtggtccctggtgctacacgacaaatccaagaaaactttacgactactgtgatgtccctcagtgtgcggccccttcatttgattgtgggaagcctcaagtggagccgaagaaatgtcctggaagggttgtgggggggtgtgtggcccacccacattcctggccctggcaagtcagtcttagaacaaggtttggaatgcacttctgtggaggcaccttgatatccccagagtgggtgttgactgctgcccactgcttggagaagtccccaaggccttcatcctacaaggtcatcctgggtgcacaccaagaagtgaatctcgaaccgcatgttcaggaaatagaagtgtctaggctgttcttggagcccacacgaaaagatattgccttgctaaagctaagcagtcctgccgtcatcactgacaaagtaatcccagcttgtctgccatccccaaattatgtggtcgctgaccggaccgaatgtttcatcactggctggggagaaacccaaggtacttttggagctggccttctcaaggaagcccagctccctgtgattgagaataaagtgtgcaatcgctatgagtttctgaatggaagagtccaatccaccgaactctgtgctgggcatttggccggaggcactgacagttgccagggtgacagtggaggtcctctggtttgcttcgagaaggacaaatacattttacaaggagtcacttcttggggtcttggctgtgcacgccccaataagcctggtgtctatgttcgtgtttcaaggtttgttacttggattgagggagtgatgagaaataattga

SEQ ID NO: 10 - protein, 803 aa, Homo sapiens
MRFPSIFTAVLFAASSALAAPVNTTTEDETAQIPAEAVIGYSDLEGDFDVAVLPFSNSTNNGLLFINTTIASIAAKEEGVSLEKREAEAKVYLSECKTGNGKNYRGTMSKTKNGITCQKWSSTSPHRPRFSPATHPSEGLEENYCRNPDNDPQGPWCYTTDPEKRYDYCDILECEEECMHCSGENYDGKISKTMSGLECQAWDSQSPHAHGYIPSKFPNKNLKKNYCRNPDRELRPWCFTTDPNKRWELCDIPRCTTPPPSSGPTYQCLKGTGENYRGNVAVTVSGHTCQHWSAQTPHTHNRTPENFPCKNLDENYCRNPDGKRAPWCHTTNSQVRWEYCKIPSCDSSPVSTEQLAPTAPPELTPVVQDCYHGDGQSYRGTSSTTTTGKKCQSWSSMTPHRHQKTPENYPNAGLTMNYCRNPDADKGPWCFTTDPSVRWEYCNLKKCSGTEASVVAPPPVVLLPDVETPSEEDCMFGNGKGYRGKRATTVTGTPCQDWAAQEPHRHSIFTPETNPRAGLEKNYCRNPDGDVGGPWCYTTNPRKLYDYCDVPQCAAPSFDCGKPQVEPKKCPGRVVGGCVAHPHSWPWQVSLRTRFGMHFCGGTLISPEWVLTAAHCLEKSPRPSSYKVILGAHQEVNLEPHVQEIEVSRLFLEPTRKDIALLKLSSPAVITDKVIPACLPSPNYVVADRTECFITGWGETQGTFGAGLLKEAQLPVIENKVCNRYEFLNGRVQSTELCAGHLAGGTDSCQGDSGGPLVCFEKDKYILQGVTSWGLGCARPNKPGVYVRVSRFVTWIEGVMRNN

SEQ ID NO: 11 - DNA, 2433 bp, Homo sapiens
atggaacataaggaagtggttcttctacttcttttatttctgaaatcaggtcaaggagagcctctggatgactatgtgaatacccagggggcttcactgttcagtgtcactaagaagcagctgggagcaggaagtatagaagaatgtgcagcaaaatgtgaggaggacgaagaattcacctgcagggcattccaatatcacagtaaagagcaacaatgtgtgataatggctgaaaacaggaagtcctccataatcattaggatgagagatgtagttttatttgaaaagaaagtgtatctctcagagtgcaagactgggaatggaaagaactacagagggacgatgtccaaaacaaaaaatggcatcacctgtcaaaaatggagttccacttctccccacagacctagattctcacctgctacacacccctcagagggactggaggagaactactgcaggaatccagacaacgatccgcaggggccctggtgctatactactgatccagaaaagagatatgactactgcgacattcttgagtgtgaagaggaatgtatgcattgcagtggagaaaactatgacggcaaaatttccaagaccatgtctggactggaatgccaggcctgggactctcagagcccacacgctcatggatacattccttccaaatttccaaacaagaacctgaagaagaattactgtcgtaaccccgatagggagctgcggccttggtgtttcaccaccgaccccaacaagcgctgggaactttgcgacatcccccgctgcacaacacctccaccatcttctggtcccacctaccagtgtctgaagggaacaggtgaaaactatcgcgggaatgtggctgttaccgtttccgggcacacctgtcagcactggagtgcacagacccctcacacacataacaggacaccagaaaacttcccctgcaaaaatttggatgaaaactactgccgcaatcctgacggaaaaagggccccatggtgccatacaaccaacagccaagtgcggtgggagtactgtaagataccgtcctgtgactcctccccagtatccacggaacaattggctcccacagcaccacctgagctaacccctgtggtccaggactgctaccatggtgatggacagagctaccgaggcacatcctccaccaccaccacaggaaagaagtgtcagtcttggtcatctatgacaccacaccggcaccagaagaccccagaaaactacccaaatgctggcctgacaatgaactactgcaggaatccagatgccgataaaggcccctggtgttttaccacagaccccagcgtcaggtgggagtactgcaacctgaaaaaatgctcaggaacagaagcgagtgttgtagcacctccgcctgttgtcctgcttccagatgtagagactccttccgaagaagactgtatgtttgggaatgggaaaggataccgaggcaagagggcgaccactgttactgggacgccatgccaggactgggctgcccaggagccccatagacacagcattttcactccagagacaaatccacgggcgggtctggaaaaaaattactgccgtaaccctgatggtgatgtaggtggtccctggtgctacacgacaaatccaagaaaactttacgactactgtgatgtccctcagtgtgcggccccttcatttgattgtgggaagcctcaagtggagccgaagaaatgtcctggaagggttgtgggggggtgtgtggcccacccacattcctggccctggcaagtcagtcttagaacaaggtttggaatgcacttctgtggaggcaccttgatatccccagagtgggtgttgactgctgcccactgcttggagaagtccccaaggccttcatcctacaaggtcatcctgggtgcacaccaagaagtgaatctcgaaccgcatgttcaggaaatagaagtgtctaggctgttcttggagcccacacgaaaagatattgccttgctaaagctaagcagtcctgccgtcatcactgacaaagtaatcccagcttgtctgccatccccaaattatgtggtcgctgaccggaccgaatgtttcatcactggctggggagaaacccaaggtacttttggagctggccttctcaaggaagcccagctccctgtgattgagaataaagtgtgcaatcgctatgagtttctgaatggaagagtccaatccaccgaactctgtgctgggcatttggccggaggcactgacagttgccagggtgacagtggaggtcctctggtttgcttcgagaaggacaaatacattttacaaggagtcacttcttggggtcttggctgtgcacgccccaataagcctggtgtctatgttcgtgtttcaaggtttgttacttggattgagggagtgatgagaaataattga

SEQ ID NO: 12 - protein, 810 aa, Homo sapiens
MEHKEVVLLLLLFLKSGQGEPLDDYVNTQGASLFSVTKKQLGAGSIEECAAKCEEDEEFTCRAFQYHSKEQQCVIMAENRKSSIIIRMRDVVLFEKKVYLSECKTGNGKNYRGTMSKTKNGITCQKWSSTSPHRPRFSPATHPSEGLEENYCRNPDNDPQGPWCYTTDPEKRYDYCDILECEEECMHCSGENYDGKISKTMSGLECQAWDSQSPHAHGYIPSKFPNKNLKKNYCRNPDRELRPWCFTTDPNKRWELCDIPRCTTPPPSSGPTYQCLKGTGENYRGNVAVTVSGHTCQHWSAQTPHTHNRTPENFPCKNLDENYCRNPDGKRAPWCHTTNSQVRWEYCKIPSCDSSPVSTEQLAPTAPPELTPVVQDCYHGDGQSYRGTSSTTTTGKKCQSWSSMTPHRHQKTPENYPNAGLTMNYCRNPDADKGPWCFTTDPSVRWEYCNLKKCSGTEASVVAPPPVVLLPDVETPSEEDCMFGNGKGYRGKRATTVTGTPCQDWAAQEPHRHSIFTPETNPRAGLEKNYCRNPDGDVGGPWCYTTNPRKLYDYCDVPQCAAPSFDCGKPQVEPKKCPGRVVGGCVAHPHSWPWQVSLRTRFGMHFCGGTLISPEWVLTAAHCLEKSPRPSSYKVILGAHQEVNLEPHVQEIEVSRLFLEPTRKDIALLKLSSPAVITDKVIPACLPSPNYVVADRTECFITGWGETQGTFGAGLLKEAQLPVIENKVCNRYEFLNGRVQSTELCAGHLAGGTDSCQGDSGGPLVCFEKDKYILQGVTSWGLGCARPNKPGVYVRVSRFVTWIEGVMRNN

SEQ ID NO: 13 - DNA, 2631 bp, Homo sapiens
atgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagagcctctggatgactatgtgaatacccagggggcttcactgttcagtgtcactaagaagcagctgggagcaggaagtatagaagaatgtgcagcaaaatgtgaggaggacgaagaattcacctgcagggcattccaatatcacagtaaagagcaacaatgtgtgataatggctgaaaacaggaagtcctccataatcattaggatgagagatgtagttttatttgaaaagaaagtgtatctctcagagtgcaagactgggaatggaaagaactacagagggacgatgtccaaaacaaaaaatggcatcacctgtcaaaaatggagttccacttctccccacagacctagattctcacctgctacacacccctcagagggactggaggagaactactgcaggaatccagacaacgatccgcaggggccctggtgctatactactgatccagaaaagagatatgactactgcgacattcttgagtgtgaagaggaatgtatgcattgcagtggagaaaactatgacggcaaaatttccaagaccatgtctggactggaatgccaggcctgggactctcagagcccacacgctcatggatacattccttccaaatttccaaacaagaacctgaagaagaattactgtcgtaaccccgatagggagctgcggccttggtgtttcaccaccgaccccaacaagcgctgggaactttgcgacatcccccgctgcacaacacctccaccatcttctggtcccacctaccagtgtctgaagggaacaggtgaaaactatcgcgggaatgtggctgttaccgtttccgggcacacctgtcagcactggagtgcacagacccctcacacacataacaggacaccagaaaacttcccctgcaaaaatttggatgaaaactactgccgcaatcctgacggaaaaagggccccatggtgccatacaaccaacagccaagtgcggtgggagtactgtaagataccgtcctgtgactcctccccagtatccacggaacaattggctcccacagcaccacctgagctaacccctgtggtccaggactgctaccatggtgatggacagagctaccgaggcacatcctccaccaccaccacaggaaagaagtgtcagtcttggtcatctatgacaccacaccggcaccagaagaccccagaaaactacccaaatgctggcctgacaatgaactactgcaggaatccagatgccgataaaggcccctggtgttttaccacagaccccagcgtcaggtgggagtactgcaacctgaaaaaatgctcaggaacagaagcgagtgttgtagcacctccgcctgttgtcctgcttccagatgtagagactccttccgaagaagactgtatgtttgggaatgggaaaggataccgaggcaagagggcgaccactgttactgggacgccatgccaggactgggctgcccaggagccccatagacacagcattttcactccagagacaaatccacgggcgggtctggaaaaaaattactgccgtaaccctgatggtgatgtaggtggtccctggtgctacacgacaaatccaagaaaactttacgactactgtgatgtccctcagtgtgcggccccttcatttgattgtgggaagcctcaagtggagccgaagaaatgtcctggaagggttgtgggggggtgtgtggcccacccacattcctggccctggcaagtcagtcttagaacaaggtttggaatgcacttctgtggaggcaccttgatatccccagagtgggtgttgactgctgcccactgcttggagaagtccccaaggccttcatcctacaaggtcatcctgggtgcacaccaagaagtgaatctcgaaccgcatgttcaggaaatagaagtgtctaggctgttcttggagcccacacgaaaagatattgccttgctaaagctaagcagtcctgccgtcatcactgacaaagtaatcccagcttgtctgccatccccaaattatgtggtcgctgaccggaccgaatgtttcatcactggctggggagaaacccaaggtacttttggagctggccttctcaaggaagcccagctccctgtgattgagaataaagtgtgcaatcgctatgagtttctgaatggaagagtccaatccaccgaactctgtgctgggcatttggccggaggcactgacagttgccagggtgacagtggaggtcctctggtttgcttcgagaaggacaaatacattttacaaggagtcacttcttggggtcttggctgtgcacgccccaataagcctggtgtctatgttcgtgtttcaaggtttgttacttggattgagggagtgatgagaaataattga

SEQ ID NO: 14 - protein, 876 aa, Homo sapiens
MRFPSIFTAVLFAASSALAAPVNTTTEDETAQIPAEAVIGYSDLEGDFDVAVLPFSNSTNNGLLFINTTIASIAAKEEGVSLEKREPLDDYVNTQGASLFSVTKKQLGAGSIEECAAKCEEDEEFTCRAFQYHSKEQQCVIMAENRKSSIIIRMRDVVLFEKKVYLSECKTGNGKNYRGTMSKTKNGITCQKWSSTSPHRPRFSPATHPSEGLEENYCRNPDNDPQGPWCYTTDPEKRYDYCDILECEEECMHCSGENYDGKISKTMSGLECQAWDSQSPHAHGYIPSKFPNKNLKKNYCRNPDRELRPWCFTTDPNKRWELCDIPRCTTPPPSSGPTYQCLKGTGENYRGNVAVTVSGHTCQHWSAQTPHTHNRTPENFPCKNLDENYCRNPDGKRAPWCHTTNSQVRWEYCKIPSCDSSPVSTEQLAPTAPPELTPVVQDCYHGDGQSYRGTSSTTTTGKKCQSWSSMTPHRHQKTPENYPNAGLTMNYCRNPDADKGPWCFTTDPSVRWEYCNLKKCSGTEASVVAPPPVVLLPDVETPSEEDCMFGNGKGYRGKRATTVTGTPCQDWAAQEPHRHSIFTPETNPRAGLEKNYCRNPDGDVGGPWCYTTNPRKLYDYCDVPQCAAPSFDCGKPQVEPKKCPGRVVGGCVAHPHSWPWQVSLRTRFGMHFCGGTLISPEWVLTAAHCLEKSPRPSSYKVILGAHQEVNLEPHVQEIEVSRLFLEPTRKDIALLKLSSPAVITDKVIPACLPSPNYVVADRTECFITGWGETQGTFGAGLLKEAQLPVIENKVCNRYEFLNGRVQSTELCAGHLAGGTDSCQGDSGGPLVCFEKDKYILQGVTSWGLGCARPNKPGVYVRVSRFVTWIEGVMRNN

SEQ ID NO: 15 - DNA, 2643 bp, Homo sapiens
atgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagctgagcctctggatgactatgtgaatacccagggggcttcactgttcagtgtcactaagaagcagctgggagcaggaagtatagaagaatgtgcagcaaaatgtgaggaggacgaagaattcacctgcagggcattccaatatcacagtaaagagcaacaatgtgtgataatggctgaaaacaggaagtcctccataatcattaggatgagagatgtagttttatttgaaaagaaagtgtatctctcagagtgcaagactgggaatggaaagaactacagagggacgatgtccaaaacaaaaaatggcatcacctgtcaaaaatggagttccacttctccccacagacctagattctcacctgctacacacccctcagagggactggaggagaactactgcaggaatccagacaacgatccgcaggggccctggtgctatactactgatccagaaaagagatatgactactgcgacattcttgagtgtgaagaggaatgtatgcattgcagtggagaaaactatgacggcaaaatttccaagaccatgtctggactggaatgccaggcctgggactctcagagcccacacgctcatggatacattccttccaaatttccaaacaagaacctgaagaagaattactgtcgtaaccccgatagggagctgcggccttggtgtttcaccaccgaccccaacaagcgctgggaactttgcgacatcccccgctgcacaacacctccaccatcttctggtcccacctaccagtgtctgaagggaacaggtgaaaactatcgcgggaatgtggctgttaccgtttccgggcacacctgtcagcactggagtgcacagacccctcacacacataacaggacaccagaaaacttcccctgcaaaaatttggatgaaaactactgccgcaatcctgacggaaaaagggccccatggtgccatacaaccaacagccaagtgcggtgggagtactgtaagataccgtcctgtgactcctccccagtatccacggaacaattggctcccacagcaccacctgagctaacccctgtggtccaggactgctaccatggtgatggacagagctaccgaggcacatcctccaccaccaccacaggaaagaagtgtcagtcttggtcatctatgacaccacaccggcaccagaagaccccagaaaactacccaaatgctggcctgacaatgaactactgcaggaatccagatgccgataaaggcccctggtgttttaccacagaccccagcgtcaggtgggagtactgcaacctgaaaaaatgctcaggaacagaagcgagtgttgtagcacctccgcctgttgtcctgcttccagatgtagagactccttccgaagaagactgtatgtttgggaatgggaaaggataccgaggcaagagggcgaccactgttactgggacgccatgccaggactgggctgcccaggagccccatagacacagcattttcactccagagacaaatccacgggcgggtctggaaaaaaattactgccgtaaccctgatggtgatgtaggtggtccctggtgctacacgacaaatccaagaaaactttacgactactgtgatgtccctcagtgtgcggccccttcatttgattgtgggaagcctcaagtggagccgaagaaatgtcctggaagggttgtgggggggtgtgtggcccacccacattcctggccctggcaagtcagtcttagaacaaggtttggaatgcacttctgtggaggcaccttgatatccccagagtgggtgttgactgctgcccactgcttggagaagtccccaaggccttcatcctacaaggtcatcctgggtgcacaccaagaagtgaatctcgaaccgcatgttcaggaaatagaagtgtctaggctgttcttggagcccacacgaaaagatattgccttgctaaagctaagcagtcctgccgtcatcactgacaaagtaatcccagcttgtctgccatccccaaattatgtggtcgctgaccggaccgaatgtttcatcactggctggggagaaacccaaggtacttttggagctggccttctcaaggaagcccagctccctgtgattgagaataaagtgtgcaatcgctatgagtttctgaatggaagagtccaatccaccgaactctgtgctgggcatttggccggaggcactgacagttgccagggtgacagtggaggtcctctggtttgcttcgagaaggacaaatacattttacaaggagtcacttcttggggtcttggctgtgcacgccccaataagcctggtgtctatgttcgtgtttcaaggtttgttacttggattgagggagtgatgagaaataattga

SEQ ID NO: 16 - protein, 880 aa, Homo sapiens [sequence omitted]

SEQ ID NO: 17 - protein, 791 aa, Homo sapiens
EPLDDYVNTQGASLFSVTKKQLGAGSIEECAAKCEEDEEFTCRAFQYHSKEQQCVIMAENRKSSIIIRMRDVVLFEKKVYLSECKTGNGKNYRGTMSKTKNGITCQKWSSTSPHRPRFSPATHPSEGLEENYCRNPDNDPQGPWCYTTDPEKRYDYCDILECEEECMHCSGENYDGKISKTMSGLECQAWDSQSPHAHGYIPSKFPNKNLKKNYCRNPDRELRPWCFTTDPNKRWELCDIPRCTTPPPSSGPTYQCLKGTGENYRGNVAVTVSGHTCQHWSAQTPHTHNRTPENFPCKNLDENYCRNPDGKRAPWCHTTNSQVRWEYCKIPSCDSSPVSTEQLAPTAPPELTPVVQDCYHGDGQSYRGTSSTTTTGKKCQSWSSMTPHRHQKTPENYPNAGLTMNYCRNPDADKGPWCFTTDPSVRWEYCNLKKCSGTEASVVAPPPVVLLPDVETPSEEDCMFGNGKGYRGKRATTVTGTPCQDWAAQEPHRHSIFTPETNPRAGLEKNYCRNPDGDVGGPWCYTTNPRKLYDYCDVPQCAAPSFDCGKPQVEPKKCPGRVVGGCVAHPHSWPWQVSLRTRFGMHFCGGTLISPEWVLTAAHCLEKSPRPSSYKVILGAHQEVNLEPHVQEIEVSRLFLEPTRKDIALLKLSSPAVITDKVIPACLPSPNYVVADRTECFITGWGETQGTFGAGLLKEAQLPVIENKVCNRYEFLNGRVQSTELCAGHLAGGTDSCQGDSGGPLVCFEKDKYILQGVTSWGLGCARPNKPGVYVRVSRFVTWIEGVMRNN

SEQ ID NO: 18 - protein, 714 aa, Homo sapiens
KVYLSECKTGNGKNYRGTMSKTKNGITCQKWSSTSPHRPRFSPATHPSEGLEENYCRNPDNDPQGPWCYTTDPEKRYDYCDILECEEECMHCSGENYDGKISKTMSGLECQAWDSQSPHAHGYIPSKFPNKNLKKNYCRNPDRELRPWCFTTDPNKRWELCDIPRCTTPPPSSGPTYQCLKGTGENYRGNVAVTVSGHTCQHWSAQTPHTHNRTPENFPCKNLDENYCRNPDGKRAPWCHTTNSQVRWEYCKIPSCDSSPVSTEQLAPTAPPELTPVVQDCYHGDGQSYRGTSSTTTTGKKCQSWSSMTPHRHQKTPENYPNAGLTMNYCRNPDADKGPWCFTTDPSVRWEYCNLKKCSGTEASVVAPPPVVLLPDVETPSEEDCMFGNGKGYRGKRATTVTGTPCQDWAAQEPHRHSIFTPETNPRAGLEKNYCRNPDGDVGGPWCYTTNPRKLYDYCDVPQCAAPSFDCGKPQVEPKKCPGRVVGGCVAHPHSWPWQVSLRTRFGMHFCGGTLISPEWVLTAAHCLEKSPRPSSYKVILGAHQEVNLEPHVQEIEVSRLFLEPTRKDIALLKLSSPAVITDKVIPACLPSPNYVVADRTECFITGWGETQGTFGAGLLKEAQLPVIENKVCNRYEFLNGRVQSTELCAGHLAGGTDSCQGDSGGPLVCFEKDKYILQGVTSWGLGCARPNKPGVYVRVSRFVTWIEGVMRNN

SEQ ID NO: 19 - DNA, 33 bp, Homo sapiens
aaaaactcgagaaaagagcacctccgcctgttg

SEQ ID NO: 20 - DNA, 45 bp, Homo sapiens
aaaaactcgagaaaagagaggctgaagctgcacctccgcctgttg

SEQ ID NO: 21 - DNA, 34 bp, Homo sapiens
aaaaactcgagaaaagaaaactttacgactactg

SEQ ID NO: 22 - DNA, 46 bp, Homo sapiens
aaaaactcgagaaaagagaggctgaagctaaactttacgactactg

SEQ ID NO: 23 - DNA, 33 bp, Homo sapiens
aaaaactcgagaaaagactttacgactactgtg

SEQ ID NO: 24 - DNA, 45 bp, Homo sapiens
aaaaactcgagaaaagagaggctgaagctctttacgactactgtg

SEQ ID NO: 25 - DNA, 36 bp, Homo sapiens
aaaaactcgagaaaagagccccttcatttgattgtg

SEQ ID NO: 26 - DNA, 48 bp, Homo sapiens
aaaaactcgagaaaagagaggctgaagctgccccttcatttgattgtg

SEQ ID NO: 27 - DNA, 37 bp, Homo sapiens
aaaaactcgagaaaagatcatttgattgtgggaagcc

SEQ ID NO: 28 - DNA, 49 bp, Homo sapiens
aaaaactcgagaaaagagaggctgaagcttcatttgattgtgggaagcc

SEQ ID NO: 29 - protein, 348 aa, Homo sapiens
APPPVVLLPDVETPSEEDCMFGNGKGYRGKRATTVTGTPCQDWAAQEPHRHSIFTPETNPRAGLEKNYCRNPDGDVGGPWCYTTNPRKLYDYCDVPQCAAPSFDCGKPQVEPKKCPGRVVGGCVAHPHSWPWQVSLRTRFGMHFCGGTLISPEWVLTAAHCLEKSPRPSSYKVILGAHQEVNLEPHVQEIEVSRLFLEPTRKDIALLKLSSPAVITDKVIPACLPSPNYVVADRTECFITGWGETQGTFGAGLLKEAQLPVIENKVCNRYEFLNGRVQSTELCAGHLAGGTDSCQGDSGGPLVCFEKDKYILQGVTSWGLGCARPNKPGVYVRVSRFVTWIEGVMRNN

SEQ ID NO: 30 - protein, 261 aa, Homo sapiens
KLYDYCDVPQCAAPSFDCGKPQVEPKKCPGRVVGGCVAHPHSWPWQVSLRTRFGMHFCGGTLISPEWVLTAAHCLEKSPRPSSYKVILGAHQEVNLEPHVQEIEVSRLFLEPTRKDIALLKLSSPAVITDKVIPACLPSPNYVVADRTECFITGWGETQGTFGAGLLKEAQLPVIENKVCNRYEFLNGRVQSTELCAGHLAGGTDSCQGDSGGPLVCFEKDKYILQGVTSWGLGCARPNKPGVYVRVSRFVTWIEGVMRNN

SEQ ID NO: 31 - protein, 260 aa, Homo sapiens
LYDYCDVPQCAAPSFDCGKPQVEPKKCPGRVVGGCVAHPHSWPWQVSLRTRFGMHFCGGTLISPEWVLTAAHCLEKSPRPSSYKVILGAHQEVNLEPHVQEIEVSRLFLEPTRKDIALLKLSSPAVITDKVIPACLPSPNYVVADRTECFITGWGETQGTFGAGLLKEAQLPVIENKVCNRYEFLNGRVQSTELCAGHLAGGTDSCQGDSGGPLVCFEKDKYILQGVTSWGLGCARPNKPGVYVRVSRFVTWIEGVMRNN

SEQ ID NO: 32 - protein, 249 aa, Homo sapiens
APSFDCGKPQVEPKKCPGRVVGGCVAHPHSWPWQVSLRTRFGMHFCGGTLISPEWVLTAAHCLEKSPRPSSYKVILGAHQEVNLEPHVQEIEVSRLFLEPTRKDIALLKLSSPAVITDKVIPACLPSPNYVVADRTECFITGWGETQGTFGAGLLKEAQLPVIENKVCNRYEFLNGRVQSTELCAGHLAGGTDSCQGDSGGPLVCFEKDKYILQGVTSWGLGCARPNKPGVYVRVSRFVTWIEGVMRNN

SEQ ID NO: 33 - protein, 247 aa, Homo sapiens
SFDCGKPQVEPKKCPGRVVGGCVAHPHSWPWQVSLRTRFGMHFCGGTLISPEWVLTAAHCLEKSPRPSSYKVILGAHQEVNLEPHVQEIEVSRLFLEPTRKDIALLKLSSPAVITDKVIPACLPSPNYVVADRTECFITGWGETQGTFGAGLLKEAQLPVIENKVCNRYEFLNGRVQSTELCAGHLAGGTDSCQGDSGGPLVCFEKDKYILQGVTSWGLGCARPNKPGVYVRVSRFVTWIEGVMRNN

SEQ ID NO: 34 - DNA, 249 bp, Homo sapiens
atgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgag

SEQ ID NO: 35 - protein, 83 aa, Homo sapiens
MRFPSIFTAVLFAASSALAAPVNTTTEDETAQIPAEAVIGYSDLEGDFDVAVLPFSNSTNNGLLFINTTIASIAAKEEGVSLE

SEQ ID NO: 36 - DNA, 6 bp, Homo sapiens
aaaaga

SEQ ID NO: 37 - DNA, 12 bp, Homo sapiens
gaggctgaagct

SEQ ID NO: 38 - protein, 2 aa, Homo sapiens
KR

SEQ ID NO: 39 - protein, 4 aa, Homo sapiens
EAEA

SEQ ID NO: 40 - protein, 433 aa, Homo sapiens
MRFPSIFTAVLFAASSALAAPVNTTTEDETAQIPAEAVIGYSDLEGDFDVAVLPFSNSTNNGLLFINTTIASIAAKEEGVSLEKRAPPPVVLLPDVETPSEEDCMFGNGKGYRGKRATTVTGTPCQDWAAQEPHRHSIFTPETNPRAGLEKNYCRNPDGDVGGPWCYTTNPRKLYDYCDVPQCAAPSFDCGKPQVEPKKCPGRVVGGCVAHPHSWPWQVSLRTRFGMHFCGGTLISPEWVLTAAHCLEKSPRPSSYKVILGAHQEVNLEPHVQEIEVSRLFLEPTRKDIALLKLSSPAVITDKVIPACLPSPNYVVADRTECFITGWGETQGTFGAGLLKEAQLPVIENKVCNRYEFLNGRVQSTELCAGHLAGGTDSCQGDSGGPLVCFEKDKYILQGVTSWGLGCARPNKPGVYVRVSRFVTWIEGVMRNN

SEQ ID NO: 41 - protein, 437 aa, Homo sapiens
MRFPSIFTAVLFAASSALAAPVNTTTEDETAQIPAEAVIGYSDLEGDFDVAVLPFSNSTNNGLLFINTTIASIAAKEEGVSLEKREAEAAPPPVVLLPDVETPSEEDCMFGNGKGYRGKRATTVTGTPCQDWAAQEPHRHSIFTPETNPRAGLEKNYCRNPDGDVGGPWCYTTNPRKLYDYCDVPQCAAPSFDCGKPQVEPKKCPGRVVGGCVAHPHSWPWQVSLRTRFGMHFCGGTLISPEWVLTAAHCLEKSPRPSSYKVILGAHQEVNLEPHVQEIEVSRLFLEPTRKDIALLKLSSPAVITDKVIPACLPSPNYVVADRTECFITGWGETQGTFGAGLLKEAQLPVIENKVCNRYEFLNGRVQSTELCAGHLAGGTDSCQGDSGGPLVCFEKDKYILQGVTSWGLGCARPNKPGVYVRVSRFVTWIEGVMRNN

SEQ ID NO: 42 - protein, 346 aa, Homo sapiens
MRFPSIFTAVLFAASSALAAPVNTTTEDETAQIPAEAVIGYSDLEGDFDVAVLPFSNSTNNGLLFINTTIASIAAKEEGVSLEKRKLYDYCDVPQCAAPSFDCGKPQVEPKKCPGRVVGGCVAHPHSWPWQVSLRTRFGMHFCGGTLISPEWVLTAAHCLEKSPRPSSYKVILGAHQEVNLEPHVQEIEVSRLFLEPTRKDIALLKLSSPAVITDKVIPACLPSPNYVVADRTECFITGWGETQGTFGAGLLKEAQLPVIENKVCNRYEFLNGRVQSTELCAGHLAGGTDSCQGDSGGPLVCFEKDKYILQGVTSWGLGCARPNKPGVYVRVSRFVTWIEGVMRNN

SEQ ID NO: 43 - protein, 350 aa, Homo sapiens
MRFPSIFTAVLFAASSALAAPVNTTTEDETAQIPAEAVIGYSDLEGDFDVAVLPFSNSTNNGLLFINTTIASIAAKEEGVSLEKREAEAKLYDYCDVPQCAAPSFDCGKPQVEPKKCPGRVVGGCVAHPHSWPWQVSLRTRFGMHFCGGTLISPEWVLTAAHCLEKSPRPSSYKVILGAHQEVNLEPHVQEIEVSRLFLEPTRKDIALLKLSSPAVITDKVIPACLPSPNYVVADRTECFITGWGETQGTFGAGLLKEAQLPVIENKVCNRYEFLNGRVQSTELCAGHLAGGTDSCQGDSGGPLVCFEKDKYILQGVTSWGLGCARPNKPGVYVRVSRFVTWIEGVMRNN

SEQ ID NO: 44 - protein, 345 aa, Homo sapiens
MRFPSIFTAVLFAASSALAAPVNTTTEDETAQIPAEAVIGYSDLEGDFDVAVLPFSNSTNNGLLFINTTIASIAAKEEGVSLEKRLYDYCDVPQCAAPSFDCGKPQVEPKKCPGRVVGGCVAHPHSWPWQVSLRTRFGMHFCGGTLISPEWVLTAAHCLEKSPRPSSYKVILGAHQEVNLEPHVQEIEVSRLFLEPTRKDIALLKLSSPAVITDKVIPACLPSPNYVVADRTECFITGWGETQGTFGAGLLKEAQLPVIENKVCNRYEFLNGRVQSTELCAGHLAGGTDSCQGDSGGPLVCFEKDKYILQGVTSWGLGCARPNKPGVYVRVSRFVTWIEGVMRNN

SEQ ID NO: 45 - protein, 349 aa, Homo sapiens
MRFPSIFTAVLFAASSALAAPVNTTTEDETAQIPAEAVIGYSDLEGDFDVAVLPFSNSTNNGLLFINTTIASIAAKEEGVSLEKREAEALYDYCDVPQCAAPSFDCGKPQVEPKKCPGRVVGGCVAHPHSWPWQVSLRTRFGMHFCGGTLISPEWVLTAAHCLEKSPRPSSYKVILGAHQEVNLEPHVQEIEVSRLFLEPTRKDIALLKLSSPAVITDKVIPACLPSPNYVVADRTECFITGWGETQGTFGAGLLKEAQLPVIENKVCNRYEFLNGRVQSTELCAGHLAGGTDSCQGDSGGPLVCFEKDKYILQGVTSWGLGCARPNKPGVYVRVSRFVTWIEGVMRNN

SEQ ID NO: 46 - protein, 334 aa, Homo sapiens
MRFPSIFTAVLFAASSALAAPVNTTTEDETAQIPAEAVIGYSDLEGDFDVAVLPFSNSTNNGLLFINTTIASIAAKEEGVSLEKRAPSFDCGKPQVEPKKCPGRVVGGCVAHPHSWPWQVSLRTRFGMHFCGGTLISPEWVLTAAHCLEKSPRPSSYKVILGAHQEVNLEPHVQEIEVSRLFLEPTRKDIALLKLSSPAVITDKVIPACLPSPNYVVADRTECFITGWGETQGTFGAGLLKEAQLPVIENKVCNRYEFLNGRVQSTELCAGHLAGGTDSCQGDSGGPLVCFEKDKYILQGVTSWGLGCARPNKPGVYVRVSRFVTWIEGVMRNN

SEQ ID NO: 47 - protein, 338 aa, Homo sapiens
MRFPSIFTAVLFAASSALAAPVNTTTEDETAQIPAEAVIGYSDLEGDFDVAVLPFSNSTNNGLLFINTTIASIAAKEEGVSLEKREAEAAPSFDCGKPQVEPKKCPGRVVGGCVAHPHSWPWQVSLRTRFGMHFCGGTLISPEWVLTAAHCLEKSPRPSSYKVILGAHQEVNLEPHVQEIEVSRLFLEPTRKDIALLKLSSPAVITDKVIPACLPSPNYVVADRTECFITGWGETQGTFGAGLLKEAQLPVIENKVCNRYEFLNGRVQSTELCAGHLAGGTDSCQGDSGGPLVCFEKDKYILQGVTSWGLGCARPNKPGVYVRVSRFVTWIEGVMRNN

SEQ ID NO: 48 - protein, 332 aa, Homo sapiens
MRFPSIFTAVLFAASSALAAPVNTTTEDETAQIPAEAVIGYSDLEGDFDVAVLPFSNSTNNGLLFINTTIASIAAKEEGVSLEKRSFDCGKPQVEPKKCPGRVVGGCVAHPHSWPWQVSLRTRFGMHFCGGTLISPEWVLTAAHCLEKSPRPSSYKVILGAHQEVNLEPHVQEIEVSRLFLEPTRKDIALLKLSSPAVITDKVIPACLPSPNYVVADRTECFITGWGETQGTFGAGLLKEAQLPVIENKVCNRYEFLNGRVQSTELCAGHLAGGTDSCQGDSGGPLVCFEKDKYILQGVTSWGLGCARPNKPGVYVRVSRFVTWIEGVMRNN

SEQ ID NO: 49 - protein, 336 aa, Homo sapiens
MRFPSIFTAVLFAASSALAAPVNTTTEDETAQIPAEAVIGYSDLEGDFDVAVLPFSNSTNNGLLFINTTIASIAAKEEGVSLEKREAEASFDCGKPQVEPKKCPGRVVGGCVAHPHSWPWQVSLRTRFGMHFCGGTLISPEWVLTAAHCLEKSPRPSSYKVILGAHQEVNLEPHVQEIEVSRLFLEPTRKDIALLKLSSPAVITDKVIPACLPSPNYVVADRTECFITGWGETQGTFGAGLLKEAQLPVIENKVCNRYEFLNGRVQSTELCAGHLAGGTDSCQGDSGGPLVCFEKDKYILQGVTSWGLGCARPNKPGVYVRVSRFVTWIEGVMRNN

SEQ ID NO: 50 - DNA, 1302 bp, Homo sapiens
atgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagcacctccgcctgttgtcctgcttccagatgtagagactccttccgaagaagactgtatgtttgggaatgggaaaggataccgaggcaagagggcgaccactgttactgggacgccatgccaggactgggctgcccaggagccccatagacacagcattttcactccagagacaaatccacgggcgggtctggaaaaaaattactgccgtaaccctgatggtgatgtaggtggtccctggtgctacacgacaaatccaagaaaactttacgactactgtgatgtccctcagtgtgcggccccttcatttgattgtgggaagcctcaagtggagccgaagaaatgtcctggaagggttgtgggggggtgtgtggcccacccacattcctggccctggcaagtcagtcttagaacaaggtttggaatgcacttctgtggaggcaccttgatatccccagagtgggtgttgactgctgcccactgcttggagaagtccccaaggccttcatcctacaaggtcatcctgggtgcacaccaagaagtgaatctcgaaccgcatgttcaggaaatagaagtgtctaggctgttcttggagcccacacgaaaagatattgccttgctaaagctaagcagtcctgccgtcatcactgacaaagtaatcccagcttgtctgccatccccaaattatgtggtcgctgaccggaccgaatgtttcatcactggctggggagaaacccaaggtacttttggagctggccttctcaaggaagcccagctccctgtgattgagaataaagtgtgcaatcgctatgagtttctgaatggaagagtccaatccaccgaactctgtgctgggcatttggccggaggcactgacagttgccagggtgacagtggaggtcctctggtttgcttcgagaaggacaaatacattttacaaggagtcacttcttggggtcttggctgtgcacgccccaataagcctggtgtctatgttcgtgtttcaaggtttgttacttggattgagggagtgatgagaaataattga

SEQ ID NO: 51 - DNA, 1314 bp, Homo sapiens
atgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagctgcacctccgcctgttgtcctgcttccagatgtagagactccttccgaagaagactgtatgtttgggaatgggaaaggataccgaggcaagagggcgaccactgttactgggacgccatgccaggactgggctgcccaggagccccatagacacagcattttcactccagagacaaatccacgggcgggtctggaaaaaaattactgccgtaaccctgatggtgatgtaggtggtccctggtgctacacgacaaatccaagaaaactttacgactactgtgatgtccctcagtgtgcggccccttcatttgattgtgggaagcctcaagtggagccgaagaaatgtcctggaagggttgtgggggggtgtgtggcccacccacattcctggccctggcaagtcagtcttagaacaaggtttggaatgcacttctgtggaggcaccttgatatccccagagtgggtgttgactgctgcccactgcttggagaagtccccaaggccttcatcctacaaggtcatcctgggtgcacaccaagaagtgaatctcgaaccgcatgttcaggaaatagaagtgtctaggctgttcttggagcccacacgaaaagatattgccttgctaaagctaagcagtcctgccgtcatcactgacaaagtaatcccagcttgtctgccatccccaaattatgtggtcgctgaccggaccgaatgtttcatcactggctggggagaaacccaaggtacttttggagctggccttctcaaggaagcccagctccctgtgattgagaataaagtgtgcaatcgctatgagtttctgaatggaagagtccaatccaccgaactctgtgctgggcatttggccggaggcactgacagttgccagggtgacagtggaggtcctctggtttgcttcgagaaggacaaatacattttacaaggagtcacttcttggggtcttggctgtgcacgccccaataagcctggtgtctatgttcgtgtttcaaggtttgttacttggattgagggagtgatgagaaataattga

SEQ ID NO: 52 - DNA, 1041 bp, Homo sapiens
atgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagaaaactttacgactactgtgatgtccctcagtgtgcggccccttcatttgattgtgggaagcctcaagtggagccgaagaaatgtcctggaagggttgtgggggggtgtgtggcccacccacattcctggccctggcaagtcagtcttagaacaaggtttggaatgcacttctgtggaggcaccttgatatccccagagtgggtgttgactgctgcccactgcttggagaagtccccaaggccttcatcctacaaggtcatcctgggtgcacaccaagaagtgaatctcgaaccgcatgttcaggaaatagaagtgtctaggctgttcttggagcccacacgaaaagatattgccttgctaaagctaagcagtcctgccgtcatcactgacaaagtaatcccagcttgtctgccatccccaaattatgtggtcgctgaccggaccgaatgtttcatcactggctggggagaaacccaaggtacttttggagctggccttctcaaggaagcccagctccctgtgattgagaataaagtgtgcaatcgctatgagtttctgaatggaagagtccaatccaccgaactctgtgctgggcatttggccggaggcactgacagttgccagggtgacagtggaggtcctctggtttgcttcgagaaggacaaatacattttacaaggagtcacttcttggggtcttggctgtgcacgccccaataagcctggtgtctatgttcgtgtttcaaggtttgttacttggattgagggagtgatgagaaataattga

SEQ ID NO: 53 - DNA, 1053 bp, Homo sapiens
atgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagctaaactttacgactactgtgatgtccctcagtgtgcggccccttcatttgattgtgggaagcctcaagtggagccgaagaaatgtcctggaagggttgtgggggggtgtgtggcccacccacattcctggccctggcaagtcagtcttagaacaaggtttggaatgcacttctgtggaggcaccttgatatccccagagtgggtgttgactgctgcccactgcttggagaagtccccaaggccttcatcctacaaggtcatcctgggtgcacaccaagaagtgaatctcgaaccgcatgttcaggaaatagaagtgtctaggctgttcttggagcccacacgaaaagatattgccttgctaaagctaagcagtcctgccgtcatcactgacaaagtaatcccagcttgtctgccatccccaaattatgtggtcgctgaccggaccgaatgtttcatcactggctggggagaaacccaaggtacttttggagctggccttctcaaggaagcccagctccctgtgattgagaataaagtgtgcaatcgctatgagtttctgaatggaagagtccaatccaccgaactctgtgctgggcatttggccggaggcactgacagttgccagggtgacagtggaggtcctctggtttgcttcgagaaggacaaatacattttacaaggagtcacttcttggggtcttggctgtgcacgccccaataagcctggtgtctatgttcgtgtttcaaggtttgttacttggattgagggagtgatgagaaataattga

SEQ ID NO: 54 - DNA, 1038 bp, Homo sapiens
atgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagactttacgactactgtgatgtccctcagtgtgcggccccttcatttgattgtgggaagcctcaagtggagccgaagaaatgtcctggaagggttgtgggggggtgtgtggcccacccacattcctggccctggcaagtcagtcttagaacaaggtttggaatgcacttctgtggaggcaccttgatatccccagagtgggtgttgactgctgcccactgcttggagaagtccccaaggccttcatcctacaaggtcatcctgggtgcacaccaagaagtgaatctcgaaccgcatgttcaggaaatagaagtgtctaggctgttcttggagcccacacgaaaagatattgccttgctaaagctaagcagtcctgccgtcatcactgacaaagtaatcccagcttgtctgccatccccaaattatgtggtcgctgaccggaccgaatgtttcatcactggctggggagaaacccaaggtacttttggagctggccttctcaaggaagcccagctccctgtgattgagaataaagtgtgcaatcgctatgagtttctgaatggaagagtccaatccaccgaactctgtgctgggcatttggccggaggcactgacagttgccagggtgacagtggaggtcctctggtttgcttcgagaaggacaaatacattttacaaggagtcacttcttggggtcttggctgtgcacgccccaataagcctggtgtctatgttcgtgtttcaaggtttgttacttggattgagggagtgatgagaaataattga

SEQ ID NO: 55 - DNA, 1050 bp, Homo sapiens
atgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagctctttacgactactgtgatgtccctcagtgtgcggccccttcatttgattgtgggaagcctcaagtggagccgaagaaatgtcctggaagggttgtgggggggtgtgtggcccacccacattcctggccctggcaagtcagtcttagaacaaggtttggaatgcacttctgtggaggcaccttgatatccccagagtgggtgttgactgctgcccactgcttggagaagtccccaaggccttcatcctacaaggtcatcctgggtgcacaccaagaagtgaatctcgaaccgcatgttcaggaaatagaagtgtctaggctgttcttggagcccacacgaaaagatattgccttgctaaagctaagcagtcctgccgtcatcactgacaaagtaatcccagcttgtctgccatccccaaattatgtggtcgctgaccggaccgaatgtttcatcactggctggggagaaacccaaggtacttttggagctggccttctcaaggaagcccagctccctgtgattgagaataaagtgtgcaatcgctatgagtttctgaatggaagagtccaatccaccgaactctgtgctgggcatttggccggaggcactgacagttgccagggtgacagtggaggtcctctggtttgcttcgagaaggacaaatacattttacaaggagtcacttcttggggtcttggctgtgcacgccccaataagcctggtgtctatgttcgtgtttcaaggtttgttacttggattgagggagtgatgagaaataattga

SEQ ID NO: 56 - DNA, 1005 bp, Homo sapiens
atgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagccccttcatttgattgtgggaagcctcaagtggagccgaagaaatgtcctggaagggttgtgggggggtgtgtggcccacccacattcctggccctggcaagtcagtcttagaacaaggtttggaatgcacttctgtggaggcaccttgatatccccagagtgggtgttgactgctgcccactgcttggagaagtccccaaggccttcatcctacaaggtcatcctgggtgcacaccaagaagtgaatctcgaaccgcatgttcaggaaatagaagtgtctaggctgttcttggagcccacacgaaaagatattgccttgctaaagctaagcagtcctgccgtcatcactgacaaagtaatcccagcttgtctgccatccccaaattatgtggtcgctgaccggaccgaatgtttcatcactggctggggagaaacccaaggtacttttggagctggccttctcaaggaagcccagctccctgtgattgagaataaagtgtgcaatcgctatgagtttctgaatggaagagtccaatccaccgaactctgtgctgggcatttggccggaggcactgacagttgccagggtgacagtggaggtcctctggtttgcttcgagaaggacaaatacattttacaaggagtcacttcttggggtcttggctgtgcacgccccaataagcctggtgtctatgttcgtgtttcaaggtttgttacttggattgagggagtgatgagaaataattga

SEQ ID NO: 57 - DNA, 1017 bp, Homo sapiens
atgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagctgccccttcatttgattgtgggaagcctcaagtggagccgaagaaatgtcctggaagggttgtgggggggtgtgtggcccacccacattcctggccctggcaagtcagtcttagaacaaggtttggaatgcacttctgtggaggcaccttgatatccccagagtgggtgttgactgctgcccactgcttggagaagtccccaaggccttcatcctacaaggtcatcctgggtgcacaccaagaagtgaatctcgaaccgcatgttcaggaaatagaagtgtctaggctgttcttggagcccacacgaaaagatattgccttgctaaagctaagcagtcctgccgtcatcactgacaaagtaatcccagcttgtctgccatccccaaattatgtggtcgctgaccggaccgaatgtttcatcactggctggggagaaacccaaggtacttttggagctggccttctcaaggaagcccagctccctgtgattgagaataaagtgtgcaatcgctatgagtttctgaatggaagagtccaatccaccgaactctgtgctgggcatttggccggaggcactgacagttgccagggtgacagtggaggtcctctggtttgcttcgagaaggacaaatacattttacaaggagtcacttcttggggtcttggctgtgcacgccccaataagcctggtgtctatgttcgtgtttcaaggtttgttacttggattgagggagtgatgagaaataattga

SEQ ID NO: 58 - DNA, 999 bp, Homo sapiens
atgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagatcatttgattgtgggaagcctcaagtggagccgaagaaatgtcctggaagggttgtgggggggtgtgtggcccacccacattcctggccctggcaagtcagtcttagaacaaggtttggaatgcacttctgtggaggcaccttgatatccccagagtgggtgttgactgctgcccactgcttggagaagtccccaaggccttcatcctacaaggtcatcctgggtgcacaccaagaagtgaatctcgaaccgcatgttcaggaaatagaagtgtctaggctgttcttggagcccacacgaaaagatattgccttgctaaagctaagcagtcctgccgtcatcactgacaaagtaatcccagcttgtctgccatccccaaattatgtggtcgctgaccggaccgaatgtttcatcactggctggggagaaacccaaggtacttttggagctggccttctcaaggaagcccagctccctgtgattgagaataaagtgtgcaatcgctatgagtttctgaatggaagagtccaatccaccgaactctgtgctgggcatttggccggaggcactgacagttgccagggtgacagtggaggtcctctggtttgcttcgagaaggacaaatacattttacaaggagtcacttcttggggtcttggctgtgcacgccccaataagcctggtgtctatgttcgtgtttcaaggtttgttacttggattgagggagtgatgagaaataattga

SEQ ID NO: 59 - DNA, 1011 bp, Homo sapiens
atgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagcttcatttgattgtgggaagcctcaagtggagccgaagaaatgtcctggaagggttgtgggggggtgtgtggcccacccacattcctggccctggcaagtcagtcttagaacaaggtttggaatgcacttctgtggaggcaccttgatatccccagagtgggtgttgactgctgcccactgcttggagaagtccccaaggccttcatcctacaaggtcatcctgggtgcacaccaagaagtgaatctcgaaccgcatgttcaggaaatagaagtgtctaggctgttcttggagcccacacgaaaagatattgccttgctaaagctaagcagtcctgccgtcatcactgacaaagtaatcccagcttgtctgccatccccaaattatgtggtcgctgaccggaccgaatgtttcatcactggctggggagaaacccaaggtacttttggagctggccttctcaaggaagcccagctccctgtgattgagaataaagtgtgcaatcgctatgagtttctgaatggaagagtccaatccaccgaactctgtgctgggcatttggccggaggcactgacagttgccagggtgacagtggaggtcctctggtttgcttcgagaaggacaaatacattttacaaggagtcacttcttggggtcttggctgtgcacgccccaataagcctggtgtctatgttcgtgtttcaaggtttgttacttggattgagggagtgatgagaaataattga

SEQ ID NO: 60 - DNA, 1047 bp, Homo sapiens
gcacctccgcctgttgtcctgcttccagatgtagagactccttccgaagaagactgtatgtttgggaatgggaaaggataccgaggcaagagggcgaccactgttactgggacgccatgccaggactgggctgcccaggagccccatagacacagcattttcactccagagacaaatccacgggcgggtctggaaaaaaattactgccgtaaccctgatggtgatgtaggtggtccctggtgctacacgacaaatccaagaaaactttacgactactgtgatgtccctcagtgtgcggccccttcatttgattgtgggaagcctcaagtggagccgaagaaatgtcctggaagggttgtgggggggtgtgtggcccacccacattcctggccctggcaagtcagtcttagaacaaggtttggaatgcacttctgtggaggcaccttgatatccccagagtgggtgttgactgctgcccactgcttggagaagtccccaaggccttcatcctacaaggtcatcctgggtgcacaccaagaagtgaatctcgaaccgcatgttcaggaaatagaagtgtctaggctgttcttggagcccacacgaaaagatattgccttgctaaagctaagcagtcctgccgtcatcactgacaaagtaatcccagcttgtctgccatccccaaattatgtggtcgctgaccggaccgaatgtttcatcactggctggggagaaacccaaggtacttttggagctggccttctcaaggaagcccagctccctgtgattgagaataaagtgtgcaatcgctatgagtttctgaatggaagagtccaatccaccgaactctgtgctgggcatttggccggaggcactgacagttgccagggtgacagtggaggtcctctggtttgcttcgagaaggacaaatacattttacaaggagtcacttcttggggtcttggctgtgcacgccccaataagcctggtgtctatgttcgtgtttcaaggtttgttacttggattgagggagtgatgagaaataattga

SEQ ID NO: 61 - DNA, 786 bp, Homo sapiens
aaactttacgactactgtgatgtccctcagtgtgcggccccttcatttgattgtgggaagcctcaagtggagccgaagaaatgtcctggaagggttgtgggggggtgtgtggcccacccacattcctggccctggcaagtcagtcttagaacaaggtttggaatgcacttctgtggaggcaccttgatatccccagagtgggtgttgactgctgcccactgcttggagaagtccccaaggccttcatcctacaaggtcatcctgggtgcacaccaagaagtgaatctcgaaccgcatgttcaggaaatagaagtgtctaggctgttcttggagcccacacgaaaagatattgccttgctaaagctaagcagtcctgccgtcatcactgacaaagtaatcccagcttgtctgccatccccaaattatgtggtcgctgaccggaccgaatgtttcatcactggctggggagaaacccaaggtacttttggagctggccttctcaaggaagcccagctccctgtgattgagaataaagtgtgcaatcgctatgagtttctgaatggaagagtccaatccaccgaactctgtgctgggcatttggccggaggcactgacagttgccagggtgacagtggaggtcctctggtttgcttcgagaaggacaaatacattttacaaggagtcacttcttggggtcttggctgtgcacgccccaataagcctggtgtctatgttcgtgtttcaaggtttgttacttggattgagggagtgatgagaaataattga

SEQ ID NO: 62 - DNA, 783 bp, Homo sapiens
ctttacgactactgtgatgtccctcagtgtgcggccccttcatttgattgtgggaagcctcaagtggagccgaagaaatgtcctggaagggttgtgggggggtgtgtggcccacccacattcctggccctggcaagtcagtcttagaacaaggtttggaatgcacttctgtggaggcaccttgatatccccagagtgggtgttgactgctgcccactgcttggagaagtccccaaggccttcatcctacaaggtcatcctgggtgcacaccaagaagtgaatctcgaaccgcatgttcaggaaatagaagtgtctaggctgttcttggagcccacacgaaaagatattgccttgctaaagctaagcagtcctgccgtcatcactgacaaagtaatcccagcttgtctgccatccccaaattatgtggtcgctgaccggaccgaatgtttcatcactggctggggagaaacccaaggtacttttggagctggccttctcaaggaagcccagctccctgtgattgagaataaagtgtgcaatcgctatgagtttctgaatggaagagtccaatccaccgaactctgtgctgggcatttggccggaggcactgacagttgccagggtgacagtggaggtcctctggtttgcttcgagaaggacaaatacattttacaaggagtcacttcttggggtcttggctgtgcacgccccaataagcctggtgtctatgttcgtgtttcaaggtttgttacttggattgagggagtgatgagaaataattga

SEQ ID NO: 63 - DNA, 750 bp, Homo sapiens
gccccttcatttgattgtgggaagcctcaagtggagccgaagaaatgtcctggaagggttgtgggggggtgtgtggcccacccacattcctggccctggcaagtcagtcttagaacaaggtttggaatgcacttctgtggaggcaccttgatatccccagagtgggtgttgactgctgcccactgcttggagaagtccccaaggccttcatcctacaaggtcatcctgggtgcacaccaagaagtgaatctcgaaccgcatgttcaggaaatagaagtgtctaggctgttcttggagcccacacgaaaagatattgccttgctaaagctaagcagtcctgccgtcatcactgacaaagtaatcccagcttgtctgccatccccaaattatgtggtcgctgaccggaccgaatgtttcatcactggctggggagaaacccaaggtacttttggagctggccttctcaaggaagcccagctccctgtgattgagaataaagtgtgcaatcgctatgagtttctgaatggaagagtccaatccaccgaactctgtgctgggcatttggccggaggcactgacagttgccagggtgacagtggaggtcctctggtttgcttcgagaaggacaaatacattttacaaggagtcacttcttggggtcttggctgtgcacgccccaataagcctggtgtctatgttcgtgtttcaaggtttgttacttggattgagggagtgatgagaaataattga

SEQ ID NO: 64 - DNA, 744 bp, Homo sapiens
tcatttgattgtgggaagcctcaagtggagccgaagaaatgtcctggaagggttgtgggggggtgtgtggcccacccacattcctggccctggcaagtcagtcttagaacaaggtttggaatgcacttctgtggaggcaccttgatatccccagagtgggtgttgactgctgcccactgcttggagaagtccccaaggccttcatcctacaaggtcatcctgggtgcacaccaagaagtgaatctcgaaccgcatgttcaggaaatagaagtgtctaggctgttcttggagcccacacgaaaagatattgccttgctaaagctaagcagtcctgccgtcatcactgacaaagtaatcccagcttgtctgccatccccaaattatgtggtcgctgaccggaccgaatgtttcatcactggctggggagaaacccaaggtacttttggagctggccttctcaaggaagcccagctccctgtgattgagaataaagtgtgcaatcgctatgagtttctgaatggaagagtccaatccaccgaactctgtgctgggcatttggccggaggcactgacagttgccagggtgacagtggaggtcctctggtttgcttcgagaaggacaaatacattttacaaggagtcacttcttggggtcttggctgtgcacgccccaataagcctggtgtctatgttcgtgtttcaaggtttgttacttggattgagggagtgatgagaaataattga

SEQ ID NO: 65 - DNA, 2376 bp, Homo sapiens
gagcctctggatgactatgtgaatacccagggggcttcactgttcagtgtcactaagaagcagctgggagcaggaagtatagaagaatgtgcagcaaaatgtgaggaggacgaagaattcacctgcagggcattccaatatcacagtaaagagcaacaatgtgtgataatggctgaaaacaggaagtcctccataatcattaggatgagagatgtagttttatttgaaaagaaagtgtatctctcagagtgcaagactgggaatggaaagaactacagagggacgatgtccaaaacaaaaaatggcatcacctgtcaaaaatggagttccacttctccccacagacctagattctcacctgctacacacccctcagagggactggaggagaactactgcaggaatccagacaacgatccgcaggggccctggtgctatactactgatccagaaaagagatatgactactgcgacattcttgagtgtgaagaggaatgtatgcattgcagtggagaaaactatgacggcaaaatttccaagaccatgtctggactggaatgccaggcctgggactctcagagcccacacgctcatggatacattccttccaaatttccaaacaagaacctgaagaagaattactgtcgtaaccccgatagggagctgcggccttggtgtttcaccaccgaccccaacaagcgctgggaactttgcgacatcccccgctgcacaacacctccaccatcttctggtcccacctaccagtgtctgaagggaacaggtgaaaactatcgcgggaatgtggctgttaccgtttccgggcacacctgtcagcactggagtgcacagacccctcacacacataacaggacaccagaaaacttcccctgcaaaaatttggatgaaaactactgccgcaatcctgacggaaaaagggccccatggtgccatacaaccaacagccaagtgcggtgggagtactgtaagataccgtcctgtgactcctccccagtatccacggaacaattggctcccacagcaccacctgagctaacccctgtggtccaggactgctaccatggtgatggacagagctaccgaggcacatcctccaccaccaccacaggaaagaagtgtcagtcttggtcatctatgacaccacaccggcaccagaagaccccagaaaactacccaaatgctggcctgacaatgaactactgcaggaatccagatgccgataaaggcccctggtgttttaccacagaccccagcgtcaggtgggagtactgcaacctgaaaaaatgctcaggaacagaagcgagtgttgtagcacctccgcctgttgtcctgcttccagatgtagagactccttccgaagaagactgtatgtttgggaatgggaaaggataccgaggcaagagggcgaccactgttactgggacgccatgccaggactgggctgcccaggagccccatagacacagcattttcactccagagacaaatccacgggcgggtctggaaaaaaattactgccgtaaccctgatggtgatgtaggtggtccctggtgctacacgacaaatccaagaaaactttacgactactgtgatgtccctcagtgtgcggccccttcatttgattgtgggaagcctcaagtggagccgaagaaatgtcctggaagggttgtgggggggtgtgtggcccacccacattcctggccctggcaagtcagtcttagaacaaggtttggaatgcacttctgtggaggcaccttgatatccccagagtgggtgttgactgctgcccactgcttggagaagtccccaaggccttcatcctacaaggtcatcctgggtgcacaccaagaagtgaatctcgaaccgcatgttcaggaaatagaagtgtctaggctgttcttggagcccacacgaaaagatattgccttgctaaagctaagcagtcctgccgtcatcactgacaaagtaatcccagcttgtctgccatccccaaattatgtggtcgctgaccggaccgaatgtttcatcactggctggggagaaacccaaggtacttttggagctggccttctcaaggaagcccagctccctgtgattgagaataaagtgtgcaatcgctatgagtttctgaatggaagagtccaatccaccgaactctgtgctgggcatttggccggaggcactgacagttgccagggtgacagtggaggtcctctggtttgcttcgagaaggacaaatacattttacaaggagtcacttcttggggtcttggctgtgcacgccccaataagcctggtgtctatgttcgtgtttcaaggtttgttacttggattgagggagtgatgagaaataattga

SEQ ID NO: 66 - DNA, 2145 bp, Homo sapiens
aaagtgtatctctcagagtgcaagactgggaatggaaagaactacagagggacgatgtccaaaacaaaaaatggcatcacctgtcaaaaatggagttccacttctccccacagacctagattctcacctgctacacacccctcagagggactggaggagaactactgcaggaatccagacaacgatccgcaggggccctggtgctatactactgatccagaaaagagatatgactactgcgacattcttgagtgtgaagaggaatgtatgcattgcagtggagaaaactatgacggcaaaatttccaagaccatgtctggactggaatgccaggcctgggactctcagagcccacacgctcatggatacattccttccaaatttccaaacaagaacctgaagaagaattactgtcgtaaccccgatagggagctgcggccttggtgtttcaccaccgaccccaacaagcgctgggaactttgcgacatcccccgctgcacaacacctccaccatcttctggtcccacctaccagtgtctgaagggaacaggtgaaaactatcgcgggaatgtggctgttaccgtttccgggcacacctgtcagcactggagtgcacagacccctcacacacataacaggacaccagaaaacttcccctgcaaaaatttggatgaaaactactgccgcaatcctgacggaaaaagggccccatggtgccatacaaccaacagccaagtgcggtgggagtactgtaagataccgtcctgtgactcctccccagtatccacggaacaattggctcccacagcaccacctgagctaacccctgtggtccaggactgctaccatggtgatggacagagctaccgaggcacatcctccaccaccaccacaggaaagaagtgtcagtcttggtcatctatgacaccacaccggcaccagaagaccccagaaaactacccaaatgctggcctgacaatgaactactgcaggaatccagatgccgataaaggcccctggtgttttaccacagaccccagcgtcaggtgggagtactgcaacctgaaaaaatgctcaggaacagaagcgagtgttgtagcacctccgcctgttgtcctgcttccagatgtagagactccttccgaagaagactgtatgtttgggaatgggaaaggataccgaggcaagagggcgaccactgttactgggacgccatgccaggactgggctgcccaggagccccatagacacagcattttcactccagagacaaatccacgggcgggtctggaaaaaaattactgccgtaaccctgatggtgatgtaggtggtccctggtgctacacgacaaatccaagaaaactttacgactactgtgatgtccctcagtgtgcggccccttcatttgattgtgggaagcctcaagtggagccgaagaaatgtcctggaagggttgtgggggggtgtgtggcccacccacattcctggccctggcaagtcagtcttagaacaaggtttggaatgcacttctgtggaggcaccttgatatccccagagtgggtgttgactgctgcccactgcttggagaagtccccaaggccttcatcctacaaggtcatcctgggtgcacaccaagaagtgaatctcgaaccgcatgttcaggaaatagaagtgtctaggctgttcttggagcccacacgaaaagatattgccttgctaaagctaagcagtcctgccgtcatcactgacaaagtaatcccagcttgtctgccatccccaaattatgtggtcgctgaccggaccgaatgtttcatcactggctggggagaaacccaaggtacttttggagctggccttctcaaggaagcccagctccctgtgattgagaataaagtgtgcaatcgctatgagtttctgaatggaagagtccaatccaccgaactctgtgctgggcatttggccggaggcactgacagttgccagggtgacagtggaggtcctctggtttgcttcgagaaggacaaatacattttacaaggagtcacttcttggggtcttggctgtgcacgccccaataagcctggtgtctatgttcgtgtttcaaggtttgttacttggattgagggagtgatgagaaataattga

(19) United States
(12) Patent Application Publication
(10) Pub. No.: US 2005/0124036 A1
(43) Pub. Date: Jun. 9, 2005

(54) Method for producing recombinant proteins in micro-organisms

(76) Inventors: Rudy Susilo, Koln (DE); Hans Christian Korting, Berlin (DE); Hans Gunther Gassen, Reinheim (DE); Martin Hils, Darmstadt (DE); Ralf Pasternack, Griesheim (DE)

Correspondence Address: Gregory Turocy;Amin & Turocy, National City Center 24th Floor, 1900 East 9th Street, Cleveland, OH, 44114, US

(21) Appl. No.: 10/503,910

(22) PCT Filed: Feb. 6, 2003
(86) PCT No.: PCT/DE03/00341

Related U.S. Application Data
(60) Provisional application No. 60/357,809, filed on Feb. 21, 2002.

(30) Foreign Application Priority Data
Feb. 6, 2002 (EP) 02002716.5

(51) Int. Cl. C07H 21/04; C12P 21/04; C12N 9/48; C12N 1/18; C12N 15/74

(52) U.S. Cl. 435/69.1; 435/471; 435/212; 435/320.1; 435/254.21; 536/23.2

(57) ABSTRACT

The invention relates to a method for producing a recombinant functional plasminogen in micro-organisms, and to a method for identifying plasminogen activators. The nucleic acid sequence coding for the fuctional part of the plasminogen is fused with a nucleic acid molecule coding for at least one signal peptide. The nucleic acid molecule coding for the plasminogen and the nucleic acid molecule coding for the signal peptide are combined with codons for interfaces of proteases which ensure the separation of the signal peptide. The recombinant plasminogen or the corresponding plasmin is suitable for treating wounds which are slow to heal or not healing, by application of the enzyme in an appropriate formulation.

The human fibrinolytic system includes as central element the protease plasmin (Pm). On the one hand plasmin is capable of degrading fibrin and on the other hand of activating matrix metalloproteinases (MMPs) and growth factors, which are in turn jointly responsible for the degradation of the extracellular matrix and for wound healing.

Plasmin originates thereby from its precursor molecule, the plasminogen. Until now, two physiologic activators of plasminogen (also referred to as plasminogen activators, PA) are known. These are the tissue-type plasminogen activator (tissue-type PA; t-PA) and the urokinase-type plasminogen activator (urokinase-type PA; u-PA). In addition the system is regulated via a set of protease inhibitors, e.g. a₂-antiplasmin. The two most important biological properties of plasminogen and plasmin respectively are directly connected to the two different activators.

The so called t-PA mediated way is responsible for fibrin homeostasis, whilst the u-PA mediated way is to be highlighted in cell migration and tissue remodeling. It could be shown in particular, that in the case of u-PA deficient mice chronic, non healing wounds occur. The same does apply to mice, the genes of which for plasminogen and t-PA and u-PA respectively were deactivated. Moreover, the life time of the animals was clearly shortened, which is inter alia due to thromboses and organ collapse. An overview about the plasminogen/plasmin system was published by Desire Collen (Thrombosis and Haemostasis, 82, 1999 (1)).

The therapeutic use of plasmin is suitable for the treatment of heart attack or stroke patients, in the case of which a rapid fibrin clot dissolving is essential for the survival, and thus represents an alternative treatment to the one with plasminogen activators, which achieve the fibrin clot hydrolysis only indirectly.

The above-mentioned mouse models show that plasmin is moreover a potential therapeutic, which can be used in the treatment of non or only slow healing wounds.

Normally the activation of plasminogen by t-PA takes place only in the presence of fibrin, as after completion of the blood coagulation cascade. In absence of a substrate plasmin is almost immediately inhibited by a₂-antiplasmin. This interaction is admittedly clearly slowed via bonding of plasmin to fibrin and the fibrin clot degradation is thereby enabled.

Different strategies of plasminogen activation are used for therapy, since in case of a heart attack or a stroke, the dissolving of blood clots is frequently inevitable for the surviving of the patients. The infusion of streptokinase for example leads to a rapid recanalisation of the vessel lumina. Thereby the activation of plasminogen with streptokinase, a bacterial protein, is not based upon a proteolytic activation but on a complexation. Then this complex can activate other plasminogen molecules to plasmin.

Further on urokinase is used therapeutically, which admittedly like streptokinase cannot distinguish fibrin-bound plasminogen from free plasminogen on a molecular level. Therefore recombinant human t-PA was developed, which proved itself as superior to streptokinase in the clinical studies. But these diagnostic findings could not be confirmed by other studies.

Precisely the recombinantly produced plasminogen activators such as rt-PA (plus different derivatives), recombinant single chain urokinase-PA and recombinant staphylokinase accentuate the importance of the production systems produced with molecular-genetically methods for the production of recombinant proteins for the use in the modern therapy.

Plasminogen is the precursor molecule of the fibrinolytic enzyme plasmin. The cDNA (Malinowski et al., Biochemistry, 23, 1984 (12); Forsgren et al., FEBS Lett. 213, 1987 (2)) as well as the gene inclusive of the non coding introns (Petersen et al., J. Biol. Chem., 265, 1990 (3)) for human plasminogen were already published in the scientific literature.

Human plasminogen (hPg), the proenzyme of the serine protease plasmin, is a glycoprotein consisting of a polypeptide chain of 791 amino acids with a molecular weight of 92.000 and a theoretical isoelectric point of 7.1. The carbohydrate rate is at 2% (Collen, 1999, (1)). Plasminogen is produced in the liver, the plasma concentration is at approximately 200 mg/l (1.5-2 μM).

The molecule is divided into 7 structure domains; accounted thereto is the N-terminal preactivation peptide (Glu-1-Lys-77), five partially homologous Kringle domains and the catalytically active proteinase domain (Val-562-Asn-791; Collen, 1999 (1)). The structure motive of the catalytic triad common to all serine proteases consists of the amino acids His-603, Asp-646, and Ser-741. The Kringle domain 1 serves as recognition sequence for binding the plasminogen to fibrin (Petersen et al., 1990 (3)) and different cell surface receptors.

Among the post-translational modifications the two essential glycosylation sites Asn-289 and Thr-346, which are both localized in the Kringle domain 3, are especially to be accentuated for the function of plasminogen (activating ability via miscellaneous proteinases and streptokinase respectively, receptor binding properties). Considering this modifications two major forms of plasminogen are distinguished:
plasminogen I features the above described glycosylation pattern
plasminogen II is lacking of the modification at Asn-289

Another glycosylation site is the amino acid Ser-248. The amino acid Ser-578 can be existent in phosphorylated form.

The activation takes place in the organism via proteolytic cleavage between the amino acids Arg-561 and Val-562. Subsequently another proteolytic activation takes place between Lys-77 and Lys-78 to the Lys-78-hPg. Alternatively this bond can be initially hydrolyzed also directly in the Glu-Pg. The active plasmin Lys-78-hPm is bonded via disulphide bridges in every case. Thereby the heavy chain of the hPm (1/78-561) is responsible for the interaction with the substrates, e.g. fibrinogen and fibrin. The light chain (562-791) resulting from the C-terminus represents the catalytically active subunit.

Already known from literature is a method, which was used for recombinant production of the fibrin binding domain of the plasminogen in Pichia pastoris with a yield of 17 mg/l (Duman et al., Biotechnol Appl Biochem. 28; 39-45, 1998 (4)). The glycosylation of this domain (Kringle 1-4) could be proven by the authors. Another citation describes the production of the two domains Kringle 4 and 5 of the human plasminogen (Guan et al., Sheng Wu Gong Cheng Xue Bao, 17, 2001 (5)). The objective was to identify the domain, which can inhibit the growth of endothelic cells.

However the plasminogen domains recombinantly produced by the two working groups in Pichia pastoris do not possess the decisive catalytic domain for the physiological functionality.

Gonzalez-Gronow et al. (Biochimica et Biophysica Acta, 1039, 1990 (6)) compared to each other the expression of recombinant human plasminogen in Escherichia coli and COS-cells, a kidney cell line of apes. The microbial production in E. coli failed, what is ascribed by the authors to the inadequate glycosylation. The production of the peptide chain was successful, but in a form not capable of activation, i.e. the treatment with activators (urokinase and t-PA) did not result in active plasmin.

The absent glycosylation results in a protein, which is lacking of the important physiological functions with regard to activation ability (no detectable enzyme activity) as well as in respect of endothelic cell recognition (Gonzalez-Gronow et al., Biochimica et Biophysica Acta, 1039, 1990 (6)). Moreover the post-translational modification with the carbohydrates significantly influences the half-life in the blood of mammals.

Whereas the authors could produce functional plasminogen in COS-cells. Other authors describe the functional expression in insect cells (Whitefleet-Smith et al., Arch. Biochem. Biophys., 271, 1989 (7)). However in the use of mammal and insect cells the time-consuming and cost-intensive cultivation conditions as well as the attainable, low protein amounts are disadvantageous. Further on mammal cells are unsuitable to produce greater amounts of a proenzyme due to the intracellular expression and the proteases in the cytoplasm (Nilsen and Castellino, Protein Expression and Purification, 16, 1999 (8) and Busby et al., J. Biol. Chem., 266, 1991 (9)). Typically in the baculovirus/lepidopteran (insect cells) system the expression yields are solely in the range of 3-10 mg/ml.

In WO0250290 the recombinant production of functional mini- and micro-plasminogen in yeast was disclosed. For this the authors expressed the genes for the catalytic domain of human plasminogen with (mini-plasminogen) or without a Kringle domain (micro-plasminogen) in the host organism Pichia pastoris. The so recombinantly produced mini- and micro-plasminogen respectively was subsequently purified, processed to mini- and micro-plasmin respectively and its activity was demonstrated in the animal experiment. The claimed yield of the recombinant proteins is at 100 mg/l for mini-plasminogen and at 3 mg/l for micro-plasminogen. However the larger a protein is the more difficult is its recombinant production, what is confirmed in the disclosure of WO0250290 by the clear decrease in the yield of micro- to mini-plasminogen in the order of two decimal powers. One example of an embodiment for the expression of longer plasminogen variants such as Lys- or Glu-plasminogen was not presented.

The recombinant production of functional plasminogen in microorganisms was not yet disclosed, so that one skilled in the art can execute it.

Therefore it is the objective of the present invention to produce in a low priced method functional human plasminogen and to process it into catalytically active plasmin.

This objective is solved by a method of recombinant production for the production of plasminogen with a microorganism according to claim 1. Further solutions are mentioned in the independent claims. The dependent claims reflect preferred embodiments.

Surprisingly it was found, that the recombinant microbial production of functional Glu- or Lys-plasminogen is possible in microorganisms. Further on it was found, that the recombinant production of micro-, mini-, Lys- and Glu-plasminogen is possible in unexpected high amounts.

Subject matter of the invention is the cloning of the plasminogen gene in expression vectors, preferred of the micro- and mini-plasminogen gene and more preferred of the Glu- or Lys-plasminogen gene or in each case of a functional variant thereof and the recombinant production of functional plasminogen, preferably functional human plasminogen using molecular genetic methods. Furthermore, the invention describes the identification of proteases, which catalyze the activation of plasminogen to plasmin. The plasminogen and plasmin respectively, which is produced through this invention, is free of contaminations such as animal proteins or viruses, which naturally occur in the isolation from humans, cattle and other mammals and which can lead to side effects in the patients.

The invention is characterized by a method of recombinant production comprising at least the following step: a.) fusion of the nucleic acid sequence coding for at least the functional part of the plasminogen peptide with a nucleic acid sequence coding for at least one signal peptide, the nucleic acid sequence coding for the functional plasminogen peptide and the nucleic acid sequence coding for at least the signal peptide being coupled with codons for cleavage sites of proteases providing for the cleavage of the signal peptide. The production of therapeutical proteins is carried out increasingly with recombinant production systems. Due to cost factors it is a strive to carry out the recombinant production in microbial, especially in bacterial organisms. These systems implicate the advantage, that besides a comparatively low price production, protein yields can be achieved in the g/l-range and the recombinant proteins are not contaminated with viruses or proteins such as prions, which can be harmful to the patients. As bacterial production systems are often not capable of producing correctly folded protein, the production is frequently carried out in eukaryotic systems such as yeasts, insect cells or mammal cells in addition to the in vitro back folding of the misfolded proteins. The eukaryotic production strains and production cell lines offer the advantage, that glycosylated proteins can be produced with them. It applies especially for insect cells or mammal cells, that the recombinant protein production is very cost intensive and the yields are frequently very low. In addition they have the disadvantage, that they can be also contaminated with viruses and proteins being harmful to humans. This is not the case in using eukaryotic microorganisms. The instrumental equipment for the cultivation of eukaryotic microorganisms is comparable to the one for bacterial organisms, contaminations with mammal viruses and proteins are not present and protein yields in the g/l-range are also possible. Especially preferred is a eukaryotic host organism which is accounted to the branch of yeasts, preferably to the Ascomycota. It is further on preferred, that it is accounted to the Saccharomycotina, especially to the class of the Saccharomycetes, here especially to the order of the Saccharomycetales. According to especially preferred embodiments, the host organism is further on accounted to the family Saccharomycetaceae, here especially to the genus Pichia. Preferred eukaryotic microorganisms used according to invention are exemplary the baker's yeast Saccharomyces cerevisiae, other examples are Candida, the methanotrophic yeasts Pichia pastoris, Pichia methanolica and Hansenula polymorpha or filamentous fungi of the genus Aspergillus, such as Aspergillus niger, Aspergillus oryzae, and Aspergillus nidulans. Especially preferred is Pichia pastoris.

The method for recombinant production is further on characterized in, that a nucleic acid molecule coding for at least the functional part of plasminogen is incorporated into an expression vector for this microorganism, the nucleic acid molecule coding preferably for human plasminogen is fused with the nucleic acid molecule coding for at least one signal peptide, preferably a prepropeptide, preferably for the transport into the endoplasmatic reticulum, codons for cleavage sites of proteases providing for the cleavage of the signal sequence or the prepropeptide in the host organism are inserted between the two nucleic acid molecules. Preferably used is a nucleic acid molecule coding for human plasminogen. In addition to a nucleic acid molecule coding for human plasminogen nucleic acid molecules can be used, which code for plasminogen from other mammals. This leads to the production of plasminogen of the respective mammals. Further on the recombinant human plasminogen is formed according to the present method by overexpression and can be, if desired, secreted into the culture medium from which it can be separated from the host cells via centrifugation, filtration or sedimentation and can be subjected to the protein purification without complex cell disruption processes, which can be carried out via methods known by the skilled in the art. The activation of plasminogen into plasmin is solved by proteases, which are capable of processing plasminogen into catalytically active plasmin.

In the following terms used in the context of the present invention are defined:

“Method for recombinant production” means, that a peptide or a protein is expressed from a nucleic acid sequence, preferably a DNA-sequence, via a suitable host organism, the nucleic acid sequence was formed from a cloning and a fusion of individual nucleic acid sections.

“Cloning” shall comprise here all known cloning methods in accordance with the state of the art, however which will not be described in detail, because they belong to the self-evident tools of the one skilled in the art.

“Expression in a suitable expression system” shall comprise here all known expression methods in accordance with the state of the art, especially those, which are mentioned in the claims.

Under the “functional plasminogen-peptide part” the part of the plasminogen or plasminogen-peptide shall be understood, which can perform the biologically relevant functions of the plasminogen. These biologically relevant functions are at least the activation ability into plasmin by plasminogen activators such as for example tissue plasminogen activator, urokinase, vampire-bat plasminogen activator, streptokinase, staphylokinase, Pla-protein from Yersinia pestis etc., and the proteolytic activity, which is characterized by the hydrolysis of fibrin. The term “plasminogen activator(s)” used in the description and the examples shall refer to proteolytic as well as non-proteolytic plasminogen activators.

Additionally in the case of Glu-plasminogen it is to be understood the processing ability into Lys-plasminogen via the plasmin-catalyzed cleavage of the preactivation peptide.

The increased activation ability of plasminogen up to the factor 1000 after binding to fibrin, laminin, fibronectin, vitronectin, heparan sulfate proteoglycan, collagen type 4 and other substrates is likewise accounted to the biological functions.

Among the biologically relevant functions of plasmin, which have to be warranted after processing of the plasminogen, is to be understood the degradation of laminin, the degradation of fibronectin, of vitronectin, of heparan sulfate proteoglycan, the activation of procollagenases, the activation of promatrix metalloproteases, the activation of latent macrophage elastase, prohormones and growth factors such as the TGFβ-1 (latent transforming growth factor), VEGF (vascular endothelial growth factor) or bFGF (basic fibroblast growth factor).

Another biological function is the inhibition ability by plasmin inhibitors such as a₂-antiplasmin and a₂-macroglobulin.

Accounted to the biologically relevant functions is moreover the bonding to fibrin, laminin, fibronectin, vitronectin, heparan sulfate proteoglycan, and collagen type 4, the bonding to receptors such as the a-enolase, annexin II or amphoterin.

First of all plasminogen is formed as inactive Glu-plasminogen. This Glu-plasminogen can be converted into Lys-plasminogen by plasmin through cleavage of the so called preactivation peptide. Both is converted by tissue plasminogen activators (so in this case only through the above-mentioned proteolytic activators) through proteolytic cleavage into plasmin, which consists of subunits connected via sulphide bridges. The smaller subunit includes the proteolytic domain and the phosphorylation site, the larger subunit carries the three glycosylations and is responsible for the bonding to fibrin. Further on the glycosylations are important for the stability in plasma. Through the formation of a 1:1-complex with streptokinase or staphylokinase, plasminogen can be converted additionally into a proteolytically active enzyme, which is capable of processing plasminogen into plasmin.

According to this, functional plasminogen is plasminogen, which can be processed by plasminogen activators into proteolytically active plasmin. Further on functional plasminogen includes preferably the fibrin binding domain and can include preferably at least one of the three glycosylations.

Smallest forms of functional plasminogen are micro- and mini-plasminogen, a larger form of Lys-plasminogen. Glu-plasminogen, which still includes the preactivation peptide, is also functional plasminogen. However it is imaginable, that regions can be omitted especially within the larger chain without interfering significantly the above-mentioned functionality (inter alia proteolysis, fibrin binding).

It is self-evident for the one skilled in the art to produce different forms of the plasminogen (referred to as plasminogen derivatives in the following), which include a functional catalytic domain. Under functional it is to be understood as already described, that the plasminogen variant features proteolytic activity after activation with plasminogen activators such as streptokinase or urokinase.
the catalytic domain can comprise deletions and amino acid exchanges or can be fused with other amino acids or peptides or proteins
the large domain can comprise all of the intermediates from Glu20 to Arg580 (based on the sequence of the pre-plasminogen), which can be activated with plasminogen activators into active plasmin

As precise example shall be mentioned three forms of Lys-plasminogen:
Variant 1: N-terminal amino acid: Met88
Variant 2: N-terminale amino acid: Lys97
Variant 3: N-terminale amino acid: Val98

The plasminogen derivatives are preferably about a number of 1 to 50 amino acids shorter or longer than the corresponding micro-, mini-, Lys- or Glu-plasminogen or preferably feature an exchange of 1 to 10 amino acids, these derivatives further on exhibit the property to be activated by plasminogen activators. Between the particular micro-, mini-, Lys- or Glu-plasminogen and the corresponding plasminogen derivative there is a sequence homology (sequence match) of over 80%, preferred of over 85%, more preferred of over 90%, furthermore preferred of over 95%, especially preferred of over 98% and further especially preferred of over 99%.

Preferably the plasminogen derivatives feature the following characteristics:
the catalytic domain can comprise at least one deletion and/or at least one amino acid exchange and/or be fused with at least another amino acid or at least another peptide or at least another protein.
the large domain can comprise all of the intermediates from Glu20 to Arg580 (based on the sequence of the pre-plasminogen), which are activable with plasminogen activators into active plasmin
a plasminogen derivative features an amino acid sequence homology (match) preferred of over 80%, more preferred of over 85%, further more preferred of over 90%, especially preferred of over 95% and further especially preferred of over 99%

With “microorganism” all such life-forms are comprised, which feature only minor dimensions. Thereby shall be comprised eukaryotic as well as prokaryotic microorganisms. Especially to be mentioned would be bacteria, yeasts, fungi and viruses.

“Nucleic acid” shall comprise DNA as well as RNA, both in all imaginable configurations, e.g. in form of double stranded nucleic acid, in form of single stranded nucleic acid, combinations thereof, as well as linear or circular nucleic acids.

Under “signal sequence” is understood a peptide sequence, which is capable of warranting the transport of another peptide sequence in or across a membrane, e.g. into the endoplasmatic reticulum. Thereby exemplary a prepropeptide, a prepeptide or a propeptide can be concerned.

With “cleavage site” such points are indicated in a peptide sequence, which provide for the cleavage of a signal sequence, a prepropeptide or propeptide from the other peptide sequence or generally the cleavage of a peptide sequence into two parts in a host organism.

A “nucleic acid coding for at least one signal peptide or a prepropeptide” is a nucleic acid sequence, which codes for a peptide or a protein structure, which provides for the other polypeptide a transfection into membranes, e.g. into the endoplasmatic reticulum.

With “primer” a starter oligonucleotide is indicated. Herewith are meant short chained, single strand oligoribo- or desoxyribonucleotides, which are complementary to a region on a single strand nucleic acid molecule and can hybridize with it into a double strand. The free 3′-hydroxy end in this double strand serves as substrate for DNA-polymerases and as starting point for the polymerization reaction of the whole single strand into the double strand. The primers are especially used in the PCR, i.e. the polymerase chain reaction known to the one skilled in the art.

With “plasmid” the nucleic acid molecules are indicated, which are not integrated into the chromosome and occur in many prokaryotic and some eukaryotic microorganisms with a length of about 2 kb up to more than 200 kb.

“Ligation” is the term for the connection of the ends of two nucleic acid molecules by means of one ligase or in line with a self-ligation, i.e. via an intramolecular ring closure reaction, in which the two single strained ends of a linear DNA-molecule dimerize provided that their ends can form base pairs with each other.

“Restriction endonuclease” is the term for a class of bacterial enzymes, which cleave phosphodiester bonds within specific base sequences in both strains of a DNA-molecule.

“Electroporation” is a method of introducing nucleic acids into cells. Thereby the cell membranes of the receiver cells, which are localized in suspension and growing exponentially, are made permeable for high molecular molecules by brief electrical pulses of high field strength while exposing them to the nucleic acid solution.

Under “overexpression” is understood an augmented production of functional plasminogen by a cell in comparison to a production by the wild type of this cell. Normally an overexpression is then spoken about, when the expressed foreign gene amounts to about 1-40% of the total cellular protein of the host cell in case of intracellular production.

Under “expression vector” are to be understood such vectors, which allow the transcription of the foreign gene cloned into the vector and the subsequent translation of the formed mRNA (messenger-RNA) after incorporating into a suitable host cell. Expression vectors normally contain the control signals, which are necessary for the expression of genes in cells of prokaryotes or eukaryotes.

In the present invention promoters which are preferably inducible by methanol such as the AOX1-promoter or especially preferred constitutive promoters such as the YPT1-promoter or the GAP-promotor are used for the control of the gene expression in yeasts such as Pichia pastoris. Especially preferred is the constitutive GAP-promoter.
“AOX1” is a gene of the alcohol oxidase 1 from P. pastoris;
“GAP” is a gene of the glyceraldehyde-3-phosphate dehydrogenase from P. pastoris and
“YPT1” is a gene of a GTP-binding protein from P. pastoris.

The signal peptides of the proteins coded by the genes PHO-1, SUC-2, PHA-E or alpha-MF are frequently used for the secretory production in yeasts.
“PHO1” is a gene of the acid phosphatase from P. pastoris;
“SUC-2” is a gene of the secretory invertase from S. cerevisiae;
“PHA-E” is a gene of the acid phosphatase from Phaseolus vulgaris Agglutinis; and
“alpha-MF” is a gene of the alpha-mating factors from S. cerevisiaea.

Especially preferred are the codons for the cleavage sites of proteases and codons for the cleavage sites for the cleavage of the propeptide for the protease Kex2 or the protease Ste13. Especially preferred the connection takes place in step a) above with codons, which code for a Kex2 cleavage site and additionally two Ste13 cleavage sites. In a preferred embodiment of the present invention the nucleic acid molecule coding for the signal peptide or prepropeptide comes from yeast, especially from the yeast Saccharomyces cerevisiae. A more preferred embodiment is directed onto a nucleic acid molecule coding for the signal peptide or the prepropeptide, which codes for the signal peptide or prepropeptide of the a-factor of the yeast Saccharomyces cerevisiae. The formed fusion product described above in step a) is preferably amplified via PCR and then further on preferably purified.

In WO02/50290 the recombinant production of mini- and micro-plasminogen is disclosed with the expression vector pPICZaA suitable for yeast that contains the inducible AOX1-promoter and the prepropeptide of the yeast alpha-factor. These smaller variants of plasminogen have either absolutely no (such as micro-plasminogen) or only one Kringle domain (such as mini-plasminogen). The expression vector pPICZaA contains the cleavage sites for the proteases Kex2 and Ste13. However the Ste13 cleavage sites were deleted in the cloning of the corresponding expression vectors of mini- and micro-plasminogen.

A set of promoters is known for inducible expression systems in yeast. Hereto accounted are inter alia the AOX1-promoter, AOX2, CUP1 (Koller A, Valesco J, Subramani S., Yeast 2000: 16(7), 651-6), PHOL (EP0495208), HIS4 (U.S. Pat. No. 4,885,242), FLD1 (Shen et al., Gene 1998: 216(1), 93-10) and the XYL1-promoter (Den Haan and Van Zyl, Appl. Microbiol. Biotechnol. 2001: 57(4), 521-7).

By means of the methanol inducible AOX1-promoter the heterologous protein production can be directed selectively and a homogeneous biomass can be obtained. Before the expression of the alien protein is induced the host organisms can achieve a high growth density without selection disadvantages, which would occur in the expression of an alien protein.

Contrary to their smaller variants, which are expressed in WO02/50290 under control of the AOX1-promoter, the recombinantly produced Glu-und Lys-plasminogen in the present invention includes all five Kringle domains, what complicates their recombinant production because of the following reasons:
possible loss of the expression cassette due to growth disadvantages for the host organisms in the expression of the alien proteins;
proteolytic degradation of the expressed proteins and
low yield

The production of Glu-oder Lys-plasminogen was not disclosed in WO02/50290 due to the described disadvantages.

These difficulties were solved in the present invention inter alia in the way, that the recombinant protein includes a signal peptide, a Kex2 and at least one Ste13, preferably two Ste13 protease cleavage sites. Further on, in a preferred embodiment a glycerol feed was carried out as another carbon source between 0.1 and 10 ml/h, preferably between 0.5 and 5 ml/h, further preferred between 0.8 and 1.5 ml/h and the culture medium was buffered at a neutral pH of 7.0. Attention was paid to sufficient oxygen feed.

In a preferred embodiment attention was paid to integrate the recombinant nucleic acid not in connection to the 5′-site of the AOX1 gene but in connection to the 5′-site of the glyceraldehyde phosphate dehydrogenase gene from P. pastoris. At this a non inducible but a constitutive promoter was used. Constitutive promoters, which are active in yeast and can be used are the GAP-promoter, the YPT1-promoter (Sears et al., Yeast 1998: 14(8), 783-90), the TKL-promoter (Den Haan and Van Zyl, Appl. Microbiol. Biotechnol. 2001: 57(4), 521-7), the ACT-promoter (Kang et al., Appl. Microbiol. Biotechnol. 2001: 55(6), 734-41) and the PMA1-promoter (Yeast 2000: 16(13), 1191-203). Preferred promoters are the GAP-promoter and the YPT1-promoter. An especially preferred promoter is the GAP-promoter.

Contrary to an inducible promoter a constitutive promoter has the disadvantage, that the alien protein to be expressed is produced constitutively, so during the whole growth phase. Through this, disadvantages occur for the host cell, what is demonstrated inter alia in a slowed growth. Due to the prevailing selection pressure, host cells which have lost the recombinant expression cassette, have an advantage and can overgrow the recombinant host cells. Through this, a heterogeneous mixed population can arise, which shall be avoided. However it was surprisingly found, that the constitutive GAP-promoter enables a higher yield according to a preferred embodiment of the present invention.

While in using the AOX1-promoter Lys-plasminogen yields were obtained after 120 hours of induction of at least 17 U/l (≡1.5 mg/l), further preferred 120 U/l (≡11 mg/l), further preferred 180 U/l (≡16 mg/l), further more preferred 200 U/l (≡18 mg/l), further preferred 220 U/l (≡20 mg/l), further more preferred 240 U/l (≡22 mg/l), especially preferred 260 U/l (≡24 mg/l) and more especially preferred 280 U/l (≡25.5 mg/l), the yields were significantly higher in using a constitutive promoter, especially the GAP-promoter.

In a preferred embodiment a constitutive promoter, e.g. the GAP-promoter is operatively coupled to a nucleic acid, coding for at least the functional part of the plasminogen sequence and being fused with nucleic acid sequence coding for at least one signal peptide, the nucleic acid sequence coding for the functional plasminogen and the nucleic acid sequence coding for at least the signal peptide being coupled with codons for cleavage sites of the proteases, which provide for the cleavage of the signal peptide.

In an especially preferred embodiment a constitutive promoter, e.g. the GAP-promoter, is operatively coupled to the nucleic acid sequence of the micro-, mini-, Lys- or Glu-plasminogen, which is fused with the nucleic acid sequence of a signal peptide from the yeast.

In this regard it was surprisingly found, that the constitutive GAP-promoter according to a preferred embodiment of the present invention enables a yield, which is about 10-times higher (see example 7c, production of Lys-plasminogen, 1375 U/l, which converted results in 125 mg/l). In another preferred embodiment a glycerol feed is carried out as another carbon source between 0.1 and 10 ml/h, preferably between 0.5 and 5 ml/h, further preferred between 0.8 and 1.5 ml/h and the culture medium was buffered at a neutral pH of 7.0. Thereby the growth rate μ [1/h] reaches values between 0.002 and 0.10, preferably between 0.004 and 0.020, further preferred between 0.008 und 0.010.

In using the GAP-promoter Lys-plasminogen yields were obtained after a fermentation period of time of 250 hours of at least 660 U/l (60 mg/l), preferred 1000 U/l (≡91 mg/l), preferred 1500 U/l (≡136 mg/l), further preferred 2000 U/l (≡182 mg/ml), especially preferred 2500 U/l (≡227 mg/l), and further especially preferred 2750 U/l (≡250 mg/l).

In the recombinant production of mini- and micro-plasminogen accordingly higher yields were obtained. The yields in case of mini-plasminogen are between from 100 mg to 2 g per liter, preferred from 300 mg/1-1.5 g/l, further preferred from 400 mg/1-1 g/l, further more preferred from 500 mg/1-800 mg/l and especially preferred from 600-700 mg/l. The yields of micro-plasminogen are further at least of 10% above the ones of mini-plasminogen. Insignificantly inferior yields were obtained in the recombinant production of Glu-plasminogen in comparison to Lys-plasminogen.

The method according to invention is suitable for the production of mini-, micro-, Lys- and Glu-plasminogens. Preferred embodiments are hence centered to the recombinant production of mini-, micro-, Lys- and Glu-plasminogen, which are each coupled to a signal or prepro sequence, in an expression vector, which contains a constitutive promoter, e.g. the GAP-promoter. In a further preferred embodiment the signal sequence consists of the signal peptide or prepropeptide of the alpha-factor of the yeast Saccharomyces cerevisiae. In an especially preferred embodiment a constitutive promoter, e.g. the GAP-promoter, is operatively coupled to a nucleic acid of the sequences Seq. ID. No. 7 or 9 or one of the sequences Seq. ID. No. 13 or 15 or one of the sequences Seq. ID. No. 50 to 59 and is expressed in a suitable expression vector.

In a further preferred embodiment a constitutive promoter, e.g. the GAP-promoter, is operatively coupled to a nucleic acid, coding at least for the functional part of the plasminogen sequence. In an especially preferred embodiment a constitutive promoter, e.g. the GAP-promoter, is operatively coupled to a nucleic acid of the sequences Seq. ID. No. 13, 15, 7 and 9 or one of the sequences Seq. ID. No. 50 to 59 or the sequence Seq. ID. No. 11 and is expressed in a suitable expression vector.

Glu-plasminogen (data calculated with the program EditSeq™ (DNASTAR))
Molecular weight: 88431.67 Dalton
791 amino acids
isoelectric point: 7.121
charge at pH 7.0: 1.351
Glycosylation sites: O-268, N-308, O-365
   (the numbering refers to the pre-plasminogen consisting of 810 amino acids) ANmerkung: dieser Fehler ist in der deutschen Anmeldung ebenfalls vorhanden.

Lys-plasminogen (data calculated with the program EditSeq™ (DNASTAR))
Molecular weight: 79655.71 Dalton
741 amino acids
isoelectric point 7.492
charge at pH 7.0: 5.287
Glycosylation sites: O-268, N-308, O-365
   (the numbering refers to the pre-plasminogen consisting of 810 amino acids)

Mini-plasminogen (data calculated with the program EditSeq™ (DNASTAR))
Molecular weight: 38169.63 Dalton
348 amino acids
isoelectric point 7.203
charge at pH 7.0: 0.893
Glycosylation sites: not any

Micro-plasminogen (data calculated with the program EditSeq™ (DNASTAR))
Molecular weight: 27230.41 Dalton
249 amino acids
7.934 isoelectric point at pH 7.0: 3.733
Glycosylation sites: not any

In the following the method according to invention is described in detail.

The fusion product generated in step a) of the present invention can be implemented moreover into an expression vector suitable for microorganisms. This expression vector is preferably chosen from the group comprising pPICZaA, B and C and pPICZ A, B and C and pGAPZaA, B and C and pGAPZA, B and C and pPIC6aA, B and C and pPIC6A, B and C as well as pAO815, pPIC3.5K and pPIC9K. The introduction into the expression vector is carried out again preferably by ligation. The PCR product as well as the expression vector are preferably cut with the restriction endonucleases KspI and XhoI, before they are ligated with a T4 DNA-ligase. The ligated nucleic acid can be transformed via electroporation in an microorganism, preferably E. coli, and the DNA can be isolated from the transformed strains obtained in that way and separated via endonucleolytic cleavage preferably with XhoI or SfuI and KspI. The nucleic acid obtained in that way can be a plasmid preferably chosen from the group pMHS476.1, pSM54.2, pSM49.8, pSM82.1, und pSM58.1, pAC37.1, pJW9.1, pPLG1.1, pPLG2.1, pPLG3.2, pPLG4.2, pPLG5.3, pPLG6.1, pPLG7.1, pPLG8.3, pPLG9.1, pPLG10.1, pPLG11.2, pPLG12.1, pPLG13.1, pPLG14.2, pPLG15.1, pPLG16.3, pPLG17.2, pPLG18.1, pPLG19.2 and pPLG20.1. As primer for the above-mentioned amplification two oligonucleotide primers are used preferably chosen from the group comprising N034 (sequence ID-No. 1), N036 (sequence-ID-No. 2), N036a (sequence-ID-No. 19), N036b (sequence-ID-No. 20), N036c (sequence-ID-No. 21), N036d (sequence-ID-No. 22), N036e (sequence-ID-No. 23), N036f (sequence-ID-No. 24), N036g (sequence-ID-No. 25), N036h (sequence-ID-No. 26), N036i (sequence-ID-No. 27), N036j (sequence-ID-No. 28), N057 (sequence ID-No. 3), N037 (sequence ID-No. 4), N035 (sequence ID-No. 5) and N056 (sequence ID-No. 6).

According to the present invention the following embodiments are especially preferred:
Codons coding for the cleavage site of the protease Kex2 and the plasminogen fusion gene, which features the nucleic acid sequence shown in sequence ID-No. 7 or 13.
Codons coding for the cleavage site of the protease Kex2 and the plasminogen fusion protein, which features the amino acid sequence shown in sequence ID-No. 8 or 14.
Codons coding for the cleavage site of the protease Kex2 and the protease Ste13 and the plasminogen fusion gene, which features the nucleic acid sequence shown in sequence ID-No. 9 or 15.
Codons coding for the protease Kex2 and the protease Ste13 and the plasminogen fusion protein, which features the amino acid sequence shown in sequence ID-No. 10 or 16.

Preferably the above-mentioned plasmid, which is preferably chosen from the above-mentioned group, is transformed into a microbial host. The transformation can be carried out for example by electroporation. The microorganism used is preferably a eukaryotic microorganism, which is accounted to the branch of the fungi. Preferred microorganisms are accounted to the Ascomycota, preferred Sacchariomycotina and therefrom preferred is the class of the Saccharomycetes, further preferred the order of the Saccharomycetales, more preferred the family of the Saccharomycetaceae and therefrom especially preferred the genus Pichia, Saccharomyces, Hansenula and Aspergillus.

According to an especially preferred embodiment of the present invention the nucleic acid sequence coding at least for the functional part of the plasminogen is overexpressed from a microbial host organism transformed with the fusion product generated in the above described step a) and at least the functional part of plasminogen is secreted, preferably it is secreted into the culture medium. According to another preferred embodiment the functional part of the nucleic acid sequence of plasminogen is one of the sequences ID-No. 60, 61, 62, 63, 64, 65 or 66. According to another preferred embodiment the functional part of the nucleic acid sequence of plasminogen corresponds to the complete plasminogen sequence. Preferably a human functional plasminogen is produced with the method of recombinant production according to the present invention.

This plasminogen, which can be obtained by the method of recombinant production according to the present invention or the plasmin resulting by the influence of proteases thereof, can be used for the production of a pharmaceutic for the treatment of wounds, especially for the treatment of slow or poorly healing wounds, for the treatment of thrombotic events or for the prevention of thrombotic events.

It was detected in addition, that the produced plasminogen according to invention as well as the obtained plasmin therefrom feature anti-coagulative properties. These advantageous properties enable in addition the use of plasminogen and/or plasmin as anti-thrombotic as well as anti-coagulative active agents for the prophylaxis and/or the treatment of heart attack, thrombosis, restenosis, hypoxia, ischemia, coagulation necrosis, inflammations of the blood vessels, as well as for treatment subsequent to a heart attack, subsequent to a bypass surgery, subsequent to an angioplasty as well as subsequent to a balloon dilatation. The plasminogen can be used also for the thrombolytic therapy in the case of acute heart attack, for the recanalization of arteriovenous shunts as well as for the reperfusion of occluded coronary arteries in the case of acute heart attack. Further uses of the produced plasminogen according to invention comprise the prophylaxis and treatment of acute lung embolism, of fresh or older coagulations of venous thromboses, acute and subacute arterial thromboses, venous thromboses, acute arterial occlusions of the extremities, chronic occlusive arteriopathies, thrombosis of arteriovenous shunts, deep venous thromboses of the hip and the extremities, early thromboses in the area of desobliterated vessels, acute central vessel occlusion at the eye, conjunctivitis in case of plasminogen type-I deficiency, burn injuries and frostbites, alkali or acid burns as well as disseminated intravasal coagulation during shock.

In case of these indications plasminogen and/or plasmin are used together preferably with an anticoagulant. As anticoagulants are suitable heparin, heparin derivatives or acetylsalicylic acid.

The present invention is hence also centered to pharmaceutical compositions, comprising a plasminogen, which was produced according to the method of recombinant production of the present invention, or the plasmin obtained therefrom, in combination with a pharmaceutically acceptable substrate, additive and/or solvent, where required. In addition the pharmaceutical compositions can contain preferably an anticoagulative active agent, especially heparin, heparin derivatives or acetylsalicylic acid.

The plasminogen produced according to invention and/or the plasmin obtainable therefrom are used in the external treatment of wounds preferably in pharmaceutical compositions, which are suitable for the topic application. Thereby plasminogen and/or plasmin are used in a concentration of 0.01-500 Upper gramme of pharmaceutical composition, preferred 0.1-500 U, further preferred 0.1-250 U, further more preferred 0.5-250 Upper gramme of pharmaceutical composition and especially preferred in a concentration of 1-150 U of plasminogen and/or plasmin per gramme of pharmaceutical composition. If plasters or other materials for dressings are used instead of semi solid formulations in form of for example ointments, pastes, gels etc., the above given concentration regions per 2 cm² of plaster surface and surface of the materials for dressings respectively are to be considered.

The pharmaceutical compositions according to invention are produced with the common solid or fluid substrates or diluents and the commonly used pharmaceutical auxiliary agents according to the desired type of application in a suitable dosage in a known way. The preferred pharmaceutical formulations or compositions are present in a pharmaceutical form, which is suitable for the local external application. Such pharmaceutical forms are for example ointments, pastes, gels, coatings, dispersions, emulsions, suspensions or special formulations, such as nanodispersed systems in form of liposomes, nanoemulsions or lipid nanoparticles, as well as tenside free formulations, polymer stabilized or particulate stabilized emulsions.

Methods for the production of diverse formulations as well as the different application methods are known to the one skilled in the art and are described in detail for example in “Remington's Pharmaceutical Sciences, Mack Publishing Co., Easton Pa.”.

The compositions produced for the parenteral application are suitable in case of using the pharmaceutical compositions for the prophylaxis and/or treatment of heart attack, thrombosis, restenosis, hypoxia, ischemia, coagulation necrosis, inflammations of the blood vessels, acute heart attack as well as for treatment subsequent to a heart attack, subsequent to a bypass surgery, subsequent to an angioplasty as well as subsequent to a balloon dilatation.

In addition the pharmaceutical compositions are suitable in case of diverse systemic applications comprising the use in case of acute lung embolism, thrombolytic therapy in the case of acute heart attack, fresh or older coagulations of venous thromboses, acute and subacute arterial thromboses, recanalization of arteriovenous shunts, venous thromboses, reperfusion of occluded coronary arteries in the case of acute heart attack, acute arterial occlusions of the extremities, chronic occlusive arteriopathies, thrombosis of arteriovenous shunts, deep venous thromboses of the hip and the extremities, early thromboses in the area of desobliterated vessels, acute central vessel occlusion at the eye, conjunctivitis in case of plasminogen type-1 deficiency, burn injuries, alkali or acid burns and frostbites, disseminated intravasal coagulation during shock.

Another possibility for application arises in case of plasminogen deficiency, such as the inherited or cogenital plasminogen deficiency (homozygote type-I plasminogen deficiency), which can result e.g. in conjunctivitis lignosa or thrombophilia. The possibility exists herein to treat the illness via for example intravenous administration of the recombinant plasminogen, inclusive of the forms Glu-, Lys-, mini- and micro-plasminogen as well as the derived variants thereof (Heinz et al., Klin. Monatsblatt Augenheilkunde 2002, 219(3): 156-8).

In another possibility for application a resolution of the pseudo membranes and the normalization of the respiratory passages as well as the improved healing of wounds can be achieved in case of administration of plasminogen. This application was described for a newborn child (The New England Journal of Medicine 1998, 339, 23, 1679-1686).

Thus the recombinantly produced plasminogen is used potentially together with the plasmin obtained therefrom or also just plasmin in pharmaceutical compositions, which are suitable for the prophylaxis and/or treatment of acute lung embolism, thrombolytic therapy in case of acute heart attack, fresh or older coagulations of venous thromboses, acute and subacute arterial thromboses, recanalization of arteriovenous shunts, venous thromboses, reperfusion of occluded coronary arteries in the case of acute heart attack, acute arterial occlusions of the extremities, chronic occlusive arteriopathies, thrombosis of arteriovenous shunts, deep venous thromboses of the hip and the extremities, early thromboses in the area of desobliterated vessels, acute central vessel occlusion at the eye, conjunctivitis in case of plasminogen type-I deficiency, burn injuries, alkali or acid burns and frostbites, disseminated intravasal coagulation during shock.

The plasminogen produced recombinantly according to invention is used preferably in pharmaceutical compositions, which are suitable for the topic treatment of burn injuries, frostbites, alkali or acid burns, injuries and/or wounds, especially poorly healing wounds. Therein the recombinant plasminogen is used preferably together with at least one activator (plasminogen activators such as for example urokinase or streptokinase). Another preferred possibility is to convert the plasminogen produced according to invention totally or partially before its use via an activator into plasmin and to use it in the herein described indications and formulations in form of plasmin or plasmin with plasminogen.

As parenteral applications are especially to be considered the intravenous, intravasale, intraperitoneal, subcutaneous as well as the intramuscular application. In case of the parenteral formulations especially in form of solutions for injection or infusion the protein is used in a concentration of 0.1-100 million units, preferred 10 to 100 million units per 10 ml solution, further preferred 1 to 10 million units per 10 ml solution and especially preferred 3 to 5 million units per 10 ml solution. In case of suitable formulations for the oral application the protein is used in a concentration of 0.1 to 100.000 units per gramme of formulation, preferred 100 to 80.000 units per gramme of formulation and especially preferred 1.000 to 50.000 units per gramme of formulation.

Further advantageous formulations are represented for example by protease containing plasters, dressings or other materials for dressings. These formulations are especially suitable for the topical application in case of wound healing, or for the treatment of burn injuries, frostbites, alkali or acid burns and/or injuries. The plasminogen produced recombinantly according to invention is preferably used in the pharmaceutical compositions, especially the wound healing agents, plasters as well as materials for dressings together with at least one activator (plasminogen activators such as for example urokinase or streptokinase) or converted in advance into plasmin via the above described activators and used as plasmin potentially together with plasminogen and potentially with at least one activator in and/or on the pharmaceutical compositions and formulations. Especially preferred is the use of plasminogen, preferred plasminogen with one activator, or plasmin or plasmin together with plasminogen and one activator in and/or on plasters and materials for dressings, which are suitable for the wound healing, especially for the treatment of poorly healing wounds, as well as for the treatment of burn injuries, frostbites, alkali or acid burns or other injuries.

The materials for dressings, wound healing dressings or plasters contain the plasminogen produced according to invention and/or plasmin obtained therefrom in a concentration of 0.01-500 units of plasminogen and/or plasmin per cm² of the pharmaceutical formulation, preferred 0.1 to 500 units of plasminogen and/or plasmin per cm² of dressing material and plaster respectively. Preferably the plasminogen and/or plasmin is contained in a concentration of 0.1-250 units, further more preferred 0.5-250 units and especially preferred of 1-150 units of plasminogen and/or one plasmin resulting therefrom per cm² of pharmaceutical formulation in the plaster or dressing material.

For the activation of 1 mg plasminogen urokinase is used between 100 μg and 1 ng, preferred between 10 μg and 10 ng urokinase are used. For the activation of 1 mg plasminogen streptokinase is used between 1 mg and 1 μg, preferred between 300 μg and 3 μg streptokinase are used. For the activation of 1 mg plasminogen protease from S. griseus is used between 100 μg and 10 ng, preferred between 10 μg and 100 ng protease from S. griseus are used. For the activation of 1 mg plasminogen protease VIII is used between 100 μg and 10 ng, preferred between 10 μg and 100 ng protease VIII are used.

Preferably the nucleic acid sequence coding for the functional part of the plasminogen is a DNA-sequence.

The present invention concerns moreover the following plasmids:
Plasmid pPLG1.1
Plasmid pPLG2.1
Plasmid pPLG3.2
Plasmid pPLG4.2
Plasmid pPLG5.3
Plasmid pPLG6.1
Plasmid pPLG7.1
Plasmid pPLG8.3
Plasmid pPLG9.1
Plasmid pPLG10.1
Plasmid pPLG11.2
Plasmid pPLG12.1.
Plasmid pPLG13.1
Plasmid pPLG14.2
Plasmid pPLG15.1
Plasmid pPLG16.3
Plasmid pPLG17.2
Plasmid pPLG18.1
Plasmid pPLG19.2
Plasmid pPLG20.1
Plasmid pMHS476.1 (deposit No.: DSM 14678)
Plasmid pSM54.2 (deposit No.: DSM 14682)
Plasmid pSM49.8 (deposit No.: DSM 14681)
Plasmid pSM82.1 (deposit No.: DSM 14679)
Plasmid pSM58.1 (deposit No.: DSM 14680)
Plasmid pAC37.1 (deposit No.: DSM 15369)
Plasmid pJW9.1 (deposit No.: DSM 15368).
   (The deposit numbers refer to the deposition at the German Collection of Microorganisms and Cell Cultures Ltd., Mascheroder Weg 1b, D-38124 Braunschweig.)

Further on the present invention concerns a DNA-sequence suitable for expression, which comprises the nucleic acid sequence coding at least for the functional part of plasminogen, obtainable by the method of recombinant production according to the present invention. Moreover it concerns the microbial host organism, which comprises the fusion product contained in the above described step a) and one nucleic acid sequence derived therefrom. Further the present invention concerns a vector, a DNA-molecule or an RNA-molecule, which comprises the fusion product contained in the above described step a) or one nucleic acid sequence derived therefrom.

Finally the present invention concerns also a method of screening for the identification of plasminogen activators, especially plasminogen activating proteases, whereas the functional plasminogen is used, produced according to the above described method of recombinant production. For this purpose preferably after preincubation of the proteases the resulting plasmin activity is measured with the functional plasminogen as produced according to the present invention. The resulting plasmin activity can be measured with a synthetic peptide substrate. Especially preferred the resulting plasmin activity is measured with N-tosyl-Gly-Pro-Lys-pNA.

The invention is explained in more detail by the drawings, which illustrate the following:

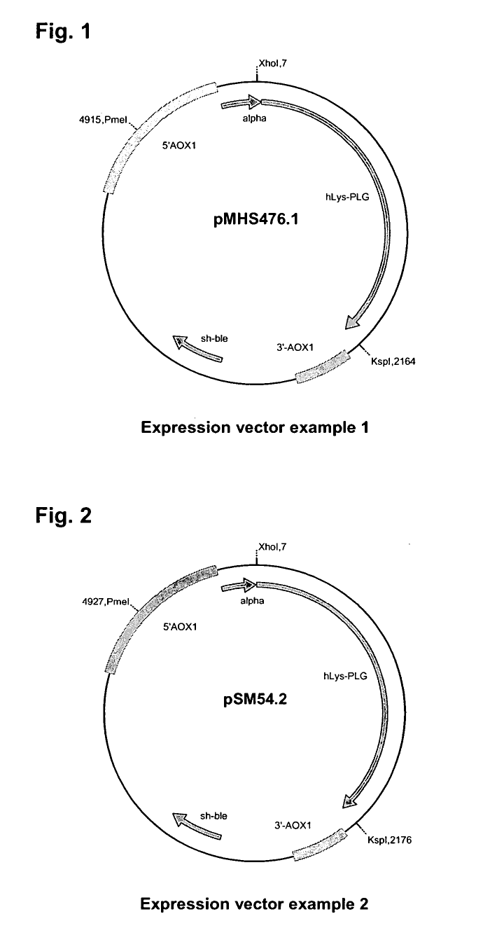

FIG. 1: Physical map of the plasmid pMHS476.1 (5682 bp). The gene of the prepropeptide of the alpha-factor is connected by the codons for a Kex2 cleavage site with the human Lys-plasminogen gene and is under the control of the AOX1-promoter.

FIG. 2: Physical map of the plasmid pSM54.2 (5694 bp). The gene of the prepropeptide of the alpha-factor is connected by the codons for a Kex2 cleavage site and two Ste13 cleavage sites with the human Lys-plasminogen gene and is under the control of the AOX1-promoter.

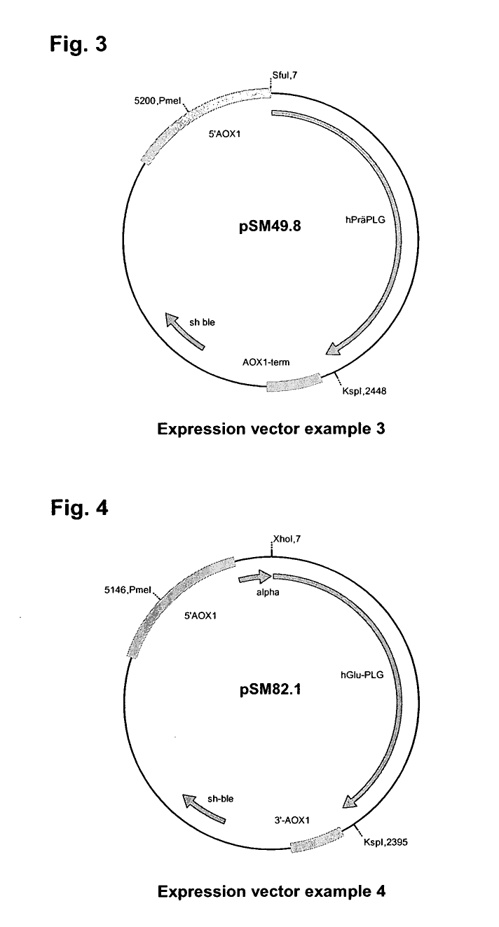

FIG. 3: Physical map of the plasmid pSM49.8 (5715 bp). The human preplasminogen gene is under the control of the AOX1-promoter.

FIG. 4: Physical map of the plasmid pSM82.1 (5913 bp). The gene of the prepropeptide of the alpha-factor is connected by the codons for a Kex2 cleavage site with the human Lys-plasminogen gene and is under the control of the AOX1-promoter.

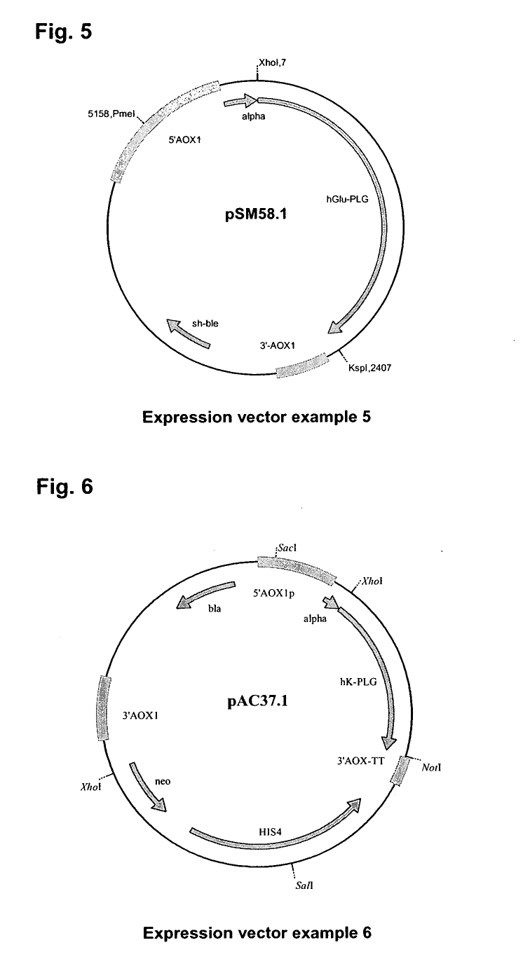

FIG. 5: Physical map of the plasmid pSM58.1 (5925 bp). The gene of the prepropeptide of the alpha-factor is connected by the codons for a Kex2 cleavage site and two Ste13 cleavage sites with the human Glu-plasminogen gene and is under the control of the AOX1-promoter.

FIG. 6: Physical map of the plasmid pAC37.1 (11400 bp). The gene of the prepropeptide of the alpha-factor is connected by the codons for a Kex2 cleavage site and two Ste13 cleavage sites with the human Lys-plasminogen gene and is under the control of the AOX1-promoter.

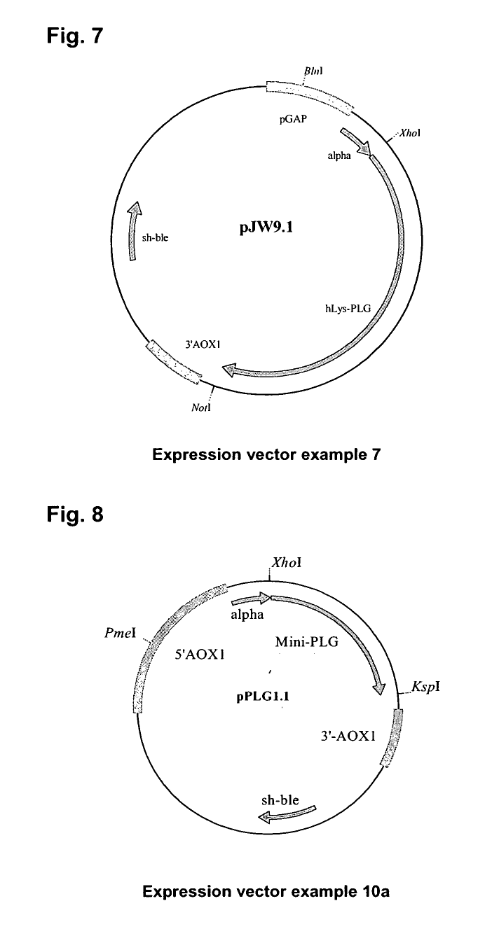

FIG. 7: Physical map of the plasmid pJW9.1 (5925 bp). The gene of the prepropeptide of the alpha-factor is connected by the codons for a Kex2 cleavage site and two Ste13 cleavage sites with the human Lys-plasminogen gene and is under the control of the GAP-promoter.

FIG. 8: Physical map of the plasmid pPLG1.1. The gene of the prepropeptide of the alpha-factor is connected by the codons for a Kex2 cleavage site and two Ste13 cleavage sites with the human mini-plasminogen gene and is under the control of the AOX1-promoter.

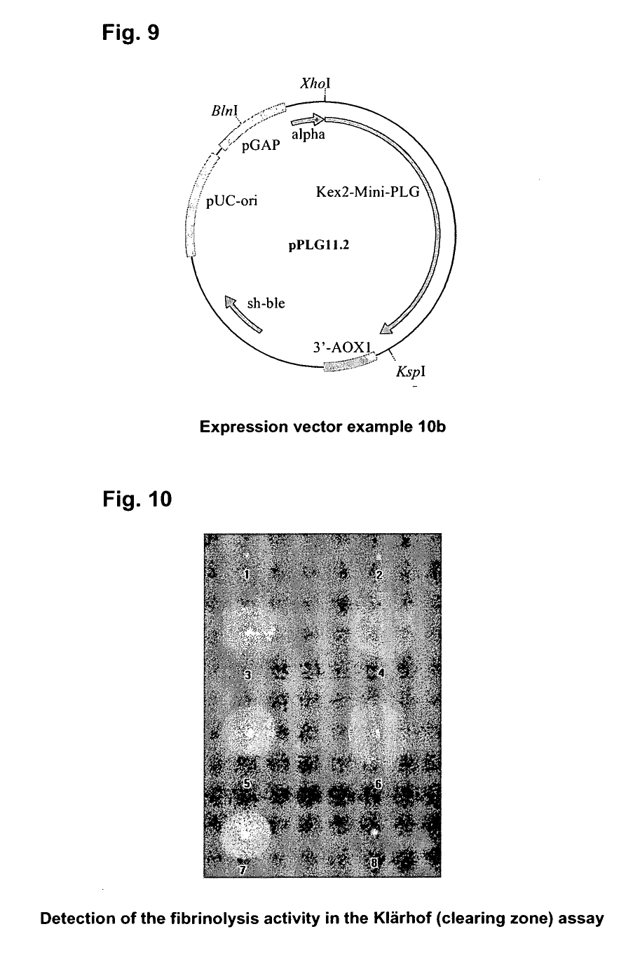

FIG. 9: Physical map of the plasmid pPLG11.2. The gene of the prepropeptide of the alpha-factor is connected by the codons for a Kex2 cleavage site and two Ste13 cleavage sites with the human mini-plasminogen gene and is under the control of the GAP-promoter.

FIG. 10: Detection of the fibrinolysis activity in the Klärhof (clearing zone) assay.

According to the invention all microorganisms can be considered as host organisms, which are capable of carrying out the glycosylation and, if desired, the secretion of proteins. Exemplary shall be mentioned here: S. cerevisiae, P. pastoris, P. methanolica and H. polymorpha or the filamentous fungus Aspergillus sp.

Especially a use of the functional plasminogen and plasmin respectively produced according to the present method of production is to be considered in a pharmaceutical formulation. In such a formulation the functional plasminogen can be mixed with a pharmaceutically acceptable substrate or auxiliary agent as well as other suitable auxiliary agents or additives in a way known to the one skilled in the art.

The Kex2 cleavage site provides for the cleavage of the propeptide by the protease Kex2 localized in the Golgi apparatus. This protease also referred to as protease YscF or as Kexin is a proprotein processing serine protease, which cuts C-terminally from basic amino acid pairs (e.g.: Lys-Arg).

The Ste13 cleavage site provides for the cleavage of the propeptide by the protease Ste13 localized in the Golgi apparatus. Ste13 (also referred to as protease YscVI or as dipeptidyl aminopeptidase A) is localized in the late Golgi and removes step by step N-terminal Xaa-Ala dipeptides, e.g. from the unripe a-factor of the yeast S. cerevisiae.

In addition to the cleavage sites for the proteases Kex2 and Ste13 other cleavage sites can be inserted, which are recognized as substrate by proteases localized in the endoplasmatic reticulum or in the Golgi apparatus.

It is also possible to fuse with the plasminogen gene exclusively a signal sequence (prepeptide) responsible for the transport into the endoplasmatic reticulum, i.e. the propeptide e.g. of the mating factor of the yeast S. cerevisiae is not necessarily required.

The microbiological, molecularbiological and protein chemical methods mentioned in the examples are well known to the one skilled in the art. The following reference books shall be mentioned as reference: Maniatis et al., Molecular Cloning: A Laboratory Manual, Cold Spring Harbor press, 1989 (10); Gassen & Schrimpf, Gentechnische Methoden, Spektrum Akademischer Verlag, Heidelberg, 1999 (11); EasySelect™ Pichia Expression Kit Instruction Manual, Invitrogen, Groningen, The Netherlands, catalog-No. K1740-01. The Pichia pastoris strains and expression systems come also from Invitrogen and are described in the above-mentioned Instruction Manual.

In case of pPICZA, B and C in short 3.3 kb Pichia pastoris expression vectors are concerned. The vectors have a zeocin resistance gene for the direct selection of Pichia transformants. Moreover the vectors have a C-terminal tag sequence, which provides for a fast purification and the detection of fusion proteins. In case of pPICZalpha A, B and C 3.6 kb Pichia pastoris expression vectors are concerned, which have also the zeocin resistance gene as well as the above-mentioned C-terminal tag sequence. In addition they contain the alpha-factor secretion signal of Saccharomyces cerevisiae for an efficient transport of proteins into the medium.

In addition the plasminogen can be activated. Thereto the plasminogen can be incubated exemplary with a protease, which was identified with the method of screening according to invention.

Preferably the plasminogen is incubated thereto with protease from S. griseus, with protease VIII or protease XVIII, with ficin, metalloendopeptidase, clostripain, with endoproteinase Glu-C, protease XIII, proteinase A, trypsin, endoproteinase Asp-N or elastase.

It is furtheron imaginable to activate plasminogen by incubation of plasminogen with one of the proteases t-PA, u-PA, or vb-PA (vampire bat-PA).

In another preferred embodiment the plasminogen is activated by incubation with staphylokinase or with streptokinase. Streptokinase or staphylokinase form with plasminogen a 1:1-complex. By this complex formation the plasminogen bound in the complex receives a conformation change, so that it becomes proteolytically active and is capable of activating plasminogen into plasmin.

The functional plasminogen or the activated functional plasminogen produced according to the present method of recombinant production is capable of hydrolysing fibrin. Further it is capable of activating promatrix metalloproteases and growth factors.

The invention will be explained in more detail by examples as follows.

### EXAMPLE 1a

### Amplification of the Lys-Plasminogen Gene with Insertion of the Codons for a Kex2 Cleavage Site at the 5′-End

The plasmid pPLGKG (Forsgren et al., FEBS Lett. 1987 Mar. 23;213(2):254-60 (2)), which contains the gene for pre-Glu-plasminogen, was isolated from the strain E. coli HB101(pPLGKG) by using the QIAGEN plasmid midi kit (QIAGEN, Hilden). 150 ng pPLGKG-DNA were linearized with 10 U of the restriction endonuclease EcoRI (Roche, Mannheim) and afterwards purified with the QIAquick PCR purification kit (QIAGEN, Hilden). For the amplification of the plasminogen gene the oligonucleotide primer pair N034 (Seq. ID No. 1) and N036 (Seq. ID No. 2) were used. The oligonucleotide primer N036 has besides the bases complementary to the plasminogen gene the codons for the Kex2 cleavage site. For the PCR were used 0.5 U Pwo-DNA-polymerase (Hybaid, Heidelberg), each with 400 nM of the oligonucleotide primer, each with 200 μM dNTP, 3 ng of linearized pPLGKG-DNA and the respective reaction buffer in a final volume of 50 μl. The primer binding temperature was 58° C.

The resulting PCR product was tested for the expected size by agarose gel electrophoresis and purified with the QIAquick PCR purification kit.

### EXAMPLE 1b

### Cloning of the Plasminogen Gene into the Vector pPICZaA

400 ng of the PCR product were cut with each 10 U of the restriction endonucleases KspI and XhoI (Roche, Mannheim). 300 ng DNA of the plasmid pPICZaA (Invitrogen, Groningen, The Netherlands), which contains the prepropeptide sequence of the α-factor from S. cerevisiae, were also cut with 10 U of the restriction endonucleases KspI and XhoI. The thus treated DNA was separated electrophoretically in a 0.9% agarose gel and the obtained fragments were extracted from the gel with the QIAquick gel extraction kit (QIAGEN, Hilden). The vector DNA was merged with the insert DNA and ligated at 4° C. over night with 1 U T4-DNA-ligase (Roche, Mannheim).

The DNA of the ligation batch was afterwards purified with the QIAquick PCR purification kit and used for the transformation of E. coli JM109 by electroporation.

The electroporated E. coli JM109 cells were incubated for 1 h at 37° C. in 1 ml SOC-medium, afterwards plated onto LB agar solid medium with 20 μg/μl zeocin (Invitrogen, Groningen, The Netherlands) and incubated at 37° C. over night.

From one of the thus obtained E. coli strains the DNA was isolated with the QIAGEN plasmid mini kit (QIAGEN, Hilden) and after endonucleolytic cleavage with the enzymes XhoI and KspI 300 ng were separated by agarose gel electrophoresis. The isolated plasmid contained a fragment of the expected size and was referred to as pMHS476.1 (FIG. 1). The correct sequence of the fusion gene from the prepropeptide gene of the alpha-factor of the yeast Saccharomyces cerevisiae and the Lys-plasminogen gene as well as the codons for the cleavage site sequence of the protease Kex2 was confirmed by sequence analysis (Seq. ID No. 7).

### EXAMPLE 1c

### Transformation of Pichia pastoris with the Plasmid pMHS476.1

With the QIAGEN plasmid midi kit plasmid-DNA of the plasminogen expression vector pMHS476.1 was isolated from the strain E. coli JM109 (pMHS476.1). 10 μg pMHS476.1-DNA were linearized with 100 U PmeI (New England Biolabs, Frankfurt) and used for the electroporation of Pichia pastoris KM71H his 4:: HIS 4 arg 4 aox1:: ARG 4 genotype of Pichia pastoris Y-11430 (Northern Regional Research Laboratories, Peoria, USA) according to the protocol shown in the EasySelect™ Pichia Expression Kit Instruction Manual. The colonies grown with 100 μg/ml zeocin after three to four days on YPDS solid medium (EasySeleCt™ Pichia Expression Kit Instruction Manual) were plated with 100 μg/ml zeocin onto YPDS solid medium and were used for the inoculation of liquid cultures. The colonies were referred to as Pichia pastoris KM71H/pMHS476.1-1/a, whereas “a” represents the consecutive numbering of the colonies beginning at 1.

### EXAMPLE 1d

### Growth of Pichia pastoris KM71H/pMHS476.1-1/1 to −1/3 and Induction of the Plasminogen Gene Expression

For the production of the precultures 100 ml BMGY-medium (EasySelect™ Pichia Expression Kit Instruction Manual) were incubated in in 1 l baffle flasks at 28° C. and 250 rpm up to a OD₆₀₀=20-30. Afterwards the precultures were centrifugated for 10 min at 4645 g and 4° C. The thus gathered cells were resuspended in BMMY-medium (0.5% methanol), so as to obtain a bio-moist mass concentration of 80 g/l. 60 ml of these main cultures were incubated for 118 h in 300 ml baffle flasks at 28° C. and 250 rpm. After 24 and 72 hours 2% methanol were added. The baffle flasks and the high revolutions per minute of 250 rpm were used to provide for a sufficient oxygen feed, which is necessary in using the AOX-promoter.

### EXAMPLE 1e

### Measurement of the Plasminogen Activity in the Supernatant of the Main Cultures After Activation with Streptokinase

The samples of the main cultures were centrifugated for 10 min at 16 000 g. 300 μl of the supernatant were incubated for 20 min at 37° C. with 1 μl streptokinase (S8026) (Sigma, Deisenhofen). To 750 μl 100 mM sodium phosphate buffer pH 8, 0.36 mM CaCl₂, 0.9% NaCl were pippeted 100 μl N-tosyl-Gly-Pro-Lys-pNA solution (9.5 mg dissolved in 75 mg glycine/10 ml, 2% Tween® 20) and incubated for 10 min at 37° C. For starting the reaction 250 μl of the supernatant pretreated with streptokinase were added and further incubated at 37° C. The extinction increase was measured photometrically at 405 nm. For the determination of control values supernatants of a parallely grown P. pastoris KM71H culture as well as supernatants without streptokinase activation were used. For the samples taken after 72 h of induction following activity values were determined (1 U/l=1 μmol N-tosyl-Gly-Pro-Lys-pNA conversion per minute per liter of culture supernatant): KM71H/pMHS476.1-1/1: 2 U/l; KM71H/pMHS476.1-1/2: 2 U/l; KM71H/pMHS476.1-1/3:1 U/l. After 118 h of induction following activity values could be determined: KM71H/pMHS476.1-1/1: 7 U/l; KM71H/pMHS476.1-1/2: 9 U/l; KM71H/pMHS476.1-1/3: 8 U/l.

### EXAMPLE 2a

### Amplification of the Lys-Plasminogen Gene with Insertion of the Codons of a Kex2 Cleavage Site and of Two Ste13 Cleavage Sites at the 5′-End

The amplification of the Lys-plasminogen gene for the cloning into the vector pPICZaA with insertion of the codons for a Kex2 cleavage site and two Ste13 cleavage sites was carried out with the two oligonucleotide primers N034 and N057 (Seq. ID No. 3) by using the conditions mentioned in example 1a. The oligonucleotide primer N057 has beside the bases complementary to the plasminogen gene the codons for the Kex2 cleavage site and the Ste13 cleavage sites.

### EXAMPLE 2b

### Cloning of the Amplified Lys-Plasminogen Gene as Described in Example 2a into the Vector pPICZaA

The cloning of the Lys-plasminogen gene into the vector pPICZaA for the production of a fusion gene from the gene of the prepropeptide of the alpha-factor of the yeast S. cerevisiae and the human plasminogen gene with insertion of the codons for the cleavage sites of the proteases Kex2 and Ste13 was carried out analogous to the cloning described in example 1b. The obtained plasmid was referred to as pSM54.2 (FIG. 2). The correct sequence (Seq. ID No. 9) was confirmed by sequence analysis.

### EXAMPLE 2c

### Transformation of Pichia pastoris with the Plasmid pSM54.2

As described for pMHS476.1 in example 1c Pichia pastoris KM71H was transformed with the plasmid pSM54.2. The obtained colonies were referred to as Pichia pastoris KM71H/pSM54.2-1/a, whereas “a” again represents the consecutive numbering of the colonies beginning at 1.

### EXAMPLE 2d

### Cultivation of Pichia pastoris KM71H/pSM54.2-1/1 to −1/3 and Induction of the Plasminogen Gene

The production of the precultures and of the main cultures as well as the induction with methanol was carried out analogous to the conditions described in example 1 d.

### EXAMPLE 2e

### Measurement of the Plasminogen Activity in the Samples of the Main Cultures after Activation with Streptokinase

The plasminogen activity after activation with streptokinase was determined as described for KM71H/pMHS476.1-1/1 to −1/3 in example 1e. For the samples taken after 72 h of induction following activity values were obtained: KM71H/pSM54.2-1/1: 2 U/l; KM71H/pSM54.2-1/2: 8 U/l; KM71H/pSM54.2-1/3: 6 U/l. After 118 h of induction following activity values could be determined: KM71H/pSM54.2-1/1: 8 U/l; KM71H/pSM54.2-1/2: 17 U/l; KM71H/pSM54.2-1/3: 13 U/l.

### EXAMPLE 3a

### Amplification of the Plasminogen Gene with Own Signal Sequence and Cloning into the Vector pPICZA; Transformation of Pichia pastoris

The amplification of the plasminogen gene inclusive of the sequence coding for the own signal peptide (pre-plasminogen) for the cloning into the vector pPICZA was carried out with the two oligonucleotide primers N034 and N037 (Seq. ID No. 4) by using the conditions described in example 1a.

The cloning of the preplasminogen gene into the vector pPICZA was carries out analogous to the cloning described in example 1b, whereas the vector as well as the PCR product were cut with the restriction endonucleases SfuI and KspI. The obtained plasmid was referred to as pSM49.8 (FIG. 3). The correct sequence (Seq. ID No. 11) was confirmed by sequence analysis.

As described for pMHS476.1 in example 1c Pichia pastoris KM71H was transformed with the plasmid pSM49.8. The obtained colonies were referred to as Pichia pastoris KM71H/pSM49.8-1/a, whereas “a” again represents the consecutive numbering of the colonies beginning at 1.

### EXAMPLE 4a

### Amplification of the Human Glu-Plasminogen Gene with Insertion of the Codons of a Kex2 Cleavage Site and Cloning into the Expression Vector pPICZα(alpha)A; Transformation of Pichia pastoris

The amplification of the Glu-plasminogen gene for the cloning into the vector pPICZaA with insertion of the codons for a Kex2 cleavage site was carried out with the two oligonucleotide primers N034 and N035 (Seq. ID No. 5) by using the conditions described in example 1a. The oligonucleotide primer N035 has beside the bases complementary to the Glu-plasminogen gene the codons for the Kex2 cleavage site.

The cloning of the Glu-plasminogen gene into the vector pPICZaA for the production of a fusion gene from the gene of the prepropeptide of the alpha-factor of the yeast S. cerevisiae and the human Glu-plasminogen gene with insertion of the codons for the cleavage sites of the protease Kex2 was carried out analogous to the cloning described in example 1b.

The obtained plasmid was referred to as pSM82.1 (FIG. 4). The correct sequence (Seq. ID No. 13) was confirmed by sequence analysis.

As described for pMHS476.1 in example 1c Pichia pastoris KM71H was transformed with the plasmid pSM82.1. The obtained colonies were referred to as Pichia pastoris KM71H/pSM82.1/a, whereas “a” again represents the consecutive numbering of the colonies beginning at 1.

### EXAMPLE 5a

### Amplification of the Human Glu-Plasminogen Gene with Insertion of the Codons of a Kex2 Cleavage Site and of Two Ste13 Cleavage Sites at the 5′-End and Cloning into the Expression Vector pPICZαA; Transformation of Pichia pastoris

The amplification of the Glu-plasminogen gene for the cloning into the vector pPICZaA with insertion of the codons for a Kex2 cleavage site and of two Ste13 cleavage sites was carried out with the two oligonucleotide primers N034 and N056 (Seq. ID No. 6) by using the conditions described in example 1a. The oligonucleotide primer N056 has beside the bases complementary to the Glu-plasminogen gene the codons for the Kex2 cleavage site and the Ste13 cleavage sites.

The cloning of the Glu-plasminogen gene into the vector pPICZaA for the production of a fusion gene from the gene of the prepropeptide of the alpha-factor of the yeast S. cerevisiae and the human Glu-plasminogen gene with insertion of the codons for the cleavage sites of the proteases Kex2 and Ste13 was carried out analogous to the cloning described in example 1b. The obtained plasmid was referred to as pSM58.1 (FIG. 5). The correct sequence (Seq. ID No. 15) was confirmed by sequence analysis

As described for pMHS476.1 in example 1c Pichia pastoris KM71H was transformed with the plasmid pSM58.1. The obtained colonies were referred to as Pichia pastoris KM71H/pSM58.1/a, whereas “a” again represents the consecutive numbering of the colonies beginning at 1.

### EXAMPLE 6a

### Insertion of the Lys-Plasminogen Gene from pSM54.2 into the Vector pPIC9K

150 ng of the vector pPIC9K (Invitrogen, Groningen, The Netherlands) were cut with each 10 U of the restriction endonucleases SacI and NotI (both Roche Diagnostics, Mannheim). 300 ng of the plasminogen expression plasmid pSM54.2 (see example 2b), were also cut with the enzymes SacI and NotI. The thus treated DNA was separated by gel electrophoresis with a 0.9% agarose gel. In each case the larger fragment was extracted from the gel by means of the QIAgen gel extraction kit (Qiagen, Hilden). The two fragments were combined and ligated at 4° C. over night with 1 U T4-DNA-ligase.

The transformation of E. coli DH5a, the isolation and the characterization of the resulting plasmid was carried out analogous to the description in example 1 b, whereas instead of the antibiotic zeocin the antibiotic ampicillin was used for the selection of transformants. The thus constructed plasmid was referred to as pAC37.1 (FIG. 6).

### EXAMPLE 6b

### Transformation of Pichia pastoris with the Plasmid pAC37.1

As described for the transformation of Pichia pastoris KM71H with pMHS476.1 in example 1c, Pichia pastoris KM71 was transformed with the plasmid pAC37.1 linearized with the restriction endonuclease SalI. The transformed cells were plated onto the histidine free medium MD-agar (Multi-Copy Pichia Expression Kit instruction manual) and incubated. The obtained colonies were referred to as Pichia pastoris KM71/pAC37.1-3/a, whereas “a” again represents the consecutive numbering of the colonies beginning at 1.

### EXAMPLE 6c

### Cultivation of Pichia pastoris KM71/pAC37.1-3/1 and Induction of the Plasminogen Gene

The production of the precultures and of the main cultures as well as the induction with methanol was carried out analogous to the conditions described in example 1d. The induction was carried out over 216 h. It was started with a methanol concentration of 0.5%, after 24 h and then in periods of 48 h 2% methanol were re-feeded.

### EXAMPLE 6d

### Measurement of the Plasminogen Activity in the Samples of the Main Cultures after Activation with Streptokinase

The plasminogen activity after activation with streptokinase was determined as described for KM71H/pMHS476.1-1/1 to −1/3 in example 1e. For the samples taken after 120 h of induction an activity of 120 U/l was obtained. After 216 h of induction an activity of 190 U/l could be measured.

### EXAMPLE 6e

### Induction of Pichia pastoris KM71/pAC37.1-3/1 in Minimal-Medium (BSM) and Measurement of the Plasminogen Activity in the Samples of the Main Cultures after Activation with Streptokinase

After the growth of Pichia pastoris KM71/pAC37.1-3/1 in BMGY-complex medium (see example 1d) 80 g of the centrifugated cells were resuspended in 100 ml of BSM-minimal medium for the induction phase. The composition of the BSM (Basal Salts Medium)-minimal medium is as follows:

H₃PO₄, 85%: 26.0 ml/l; CaCl₂.2H₂O: 0.6 g/l; K₂SO₄: 18.0 g/i; MgSO₄.7H₂O:14.0 g/l; KOH: 4.0 g/l; glycerine: 20 ml/l; antifoam: 1.0 ml/l; trace solution: 8.0 ml/l; biotin solution (0.2 g/l): 8.0 ml/l.

Composition of the trace solution: H₂SO₄: 5.0 ml/l; CuSO₄.5H₂O: 6.0 g/l; KI: 0.08 g/l; MnSO₄.H₂O: 3.0 g/l; Na₂MoO₄: 0.2 g/l; H₃BO₃: 0.02 g/l; CoCl₂: 0.5 g/l; ZnCl₂: 20.0 g/l; FeSO₄.7H₂O: 65.0 g/l.

For the induction 2% methanol were added daily. The plasminogen activity after activation with streptokinase was determined as described for KM71H/pMHS476.1-1/1 to −1/3 in example 1e. After 120 h of induction a plasminogen activity of 193 U/l was determined, after 168 h 289 U/l could me measured.

### EXAMPLE 6f

### Detection of the Plasminogen Activity in the Samples of the Main Cultures after Activation with Streptokinase in the Klärhof (Clearing Zone) Fibrinolysis Test

For the preparation of the Klärhof (clearing zone) fibrinolysis test (Stack, M. S., Pizzo, S. V., and Gonzalez-Gronow, M. (1992): Effect of desialylation on the biological properties of human plasminogen. Biochem. J. 284, 81-86) (13) 1.5 g GTG-low-melting agarose were melted by boiling up in 75 ml 50 mM sodium phosphate buffer pH 7.4. 35 ml of a fibrinogen solution (225 mg/37.5 ml 50 mM sodium phosphate buffer pH 7.4) were mixed bubble free with 350 μl thrombin solution (10 U/ml in 50 mM sodium phosphate buffer pH 7.4), stirred into the agarose solution and poured in a petri dish. After solidifying of the fibrin agar 1 mm sized holes were engraved into the agar.

For detecting the fibrinolysis activity of the recombinantly produced plasminogen after streptokinase activation in each case 20 μl of the following solutions were pipetted into the holes and incubated for 20 h at 37° C.:
1: 0.5 mg/ml plasminogen (Roche, Mannheim)
2: culture supernatant KM71/pAC37.1-3/1 from example 6e
3: 0.5 mg/ml plasminogen, activated by streptokinase
4: culture supernatant KM71/pAC37.1-3/1 from example 6e, activated by streptokinase
5: 0.25 mg/ml plasminogen, activated by streptokinase
6: culture supernatant KM71/pAC37.1-3/1 from example 6e, diluted 1:2, activated by streptokinase
7: 0.125 mg/ml plasminogen, activated by streptokinase
8: culture supernatant KM71H, produced as described in example 6e for KM71/pAC37.1-3/1, activated by streptokinase

For the activation with streptokinase 2 μl streptokinase (100 U/μl, Sigma, Deisenhofen) were pipetted to 40 μl of the respective solutions and incubated for 60 min at 37° C.

The spots obtained by fibrinolytic activity are shown in FIG. 10.

### EXAMPLE 6g

### Purification of the Plasminogen Produced Recombinantly in Pichia pastoris KM71/pAC37.1-3/1 by Affinity Chromatography

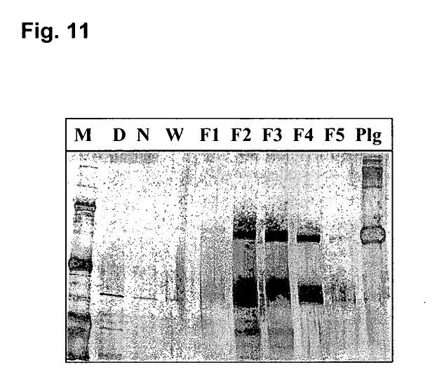

50 ml of the culture supernatant of Pichia pastoris KM71/pAC37.1-3/1 from example 6c/6d were dialyzed at 4° C. contra 4 l 50 mM sodium phosphate buffer pH 7.5. After 24 h the dialysis buffer was exchanged and dialyzed for another 24 h. The dialysate was afterwards pressed through a 0.02 μm filter and then given onto a lysine-sepharose™ 4B column (diameter: 16 mm, height: 95 mm) (Amersham Biosciences) equilibrated with 50 mM sodium phosphate buffer pH 7.5. Unspecifically bound proteins were washed off the column with 50 mM sodium phosphate buffer pH 7.5, 0.5 M NaCl. The bound plasminogen was eluted with 50 mM sodium phosphate buffer pH 7.5, 0.01 M aminocaproic acid. Individual samples were analyzed by 7.5% SDS-PAGE with subsequent silver staining (FIG. 11). The recombinant plasminogen contained in the fractions is localized in the gel on the height of the human plasminogen added as reference.

FIG. 11 shows a 7.5% SDS-PAGE of the purification fractions from example 6g. In FIG. 11 the used abbreviations have the meanings as follows:
M: size standard (from top to bottom: 116 kDa, 66 kDa, 45 kDa, 35 kDa)
D: dialysate,
N: non binding fraction,
W: washing fraction,
F1-F5 plasminogen containing elution fractions,
Plg: plasminogen (American Diagnostica, Pfungstadt)

### EXAMPLE 6h

### Fermentation of Pichia pastoris KM71/pAC37.1-3/1 for the Evaluation of the pH Value and the Substrate Influence

50 ml of YEP-G-medium (10 g/l yeast extract, 20 g/l casein peptone, 20 g/l glycerol) in a 1 l wide neck flask without baffles were inoculated with 2 ml glycerol cryo-culture Pichia pastoris KM71/pAC37.1-3/1 and incubated for 9 h at 30° C. and 300 rpm. 5 ml of this culture were used to inoculate 50 ml of MG-medium (5 g/l yeast nitrogen base w/o amino acids, 20 μl glycerol, 2.5 ml/l biotin solution (0.2g/l)) in a 1 l wide neck shaking flask without baffles. This second preculture was incubated for 16 h at 30° C. and 300 rpm. The main culture was fermented in the multi fermentation apparatus “stirrer-pro” (DASGIP, Jülich), which allows the parallel fermentation of four cultures at different conditions. Therefore in each case 150 ml BSM-medium (see example 6e) were inoculated with 15 ml of the second preculture. The fermentations were started at pH 6, the target pH value was headed for after initiating the substrate dosage. The different conditions and results of the parallel fermentations are shown in tab. 1.

TAB 1

| Exp. | pH | substrate | feed rate | OD₆₀₀ | plasminogen conc. |
|---|---|---|---|---|---|
| I | 6 | methanol | profile | 187 | 1.4 mg/l |
| II | 7 | methanol | profile | 160 | 6.1 mg/l |
| III | 6 | methanol/glycerol | 1 ml/h | 270 | 10.1 mg/l |
| IV | 6 | methanol | profile | 130 | 3.4 mg/l |

In experiment IV 30 g/l peptone was added to the medium. Before the initiation of the methanol dosage glycerol feed medium (500 g/l water free glycerol, 10 ml/l trace solution, 10 ml/l biotin solution [see example 6e]) was added for 4 h with a constant rate of 24 ml/h. For the profile in the experiments I, II and IV the following term was given in as dosage function f(x)=P1+(P2/1+exp(−P3(t-P4))))+(P5/(1+exp(−P6(t-P7)))) with P1=0; P2=0.7; P3=0.2; P4=15; P5=P6=P7=0. It can be seen from tab. 1, that the plasminogen concentrations at neutral pH value and mixed glycerol/methanol dosage are the highest.

### EXAMPLE 7a

### Insertion of the Lys-Plasminogen Gene from pAC37.1 into the Vector pGAPZαA

150 ng of the vector pGAPZaA (invitrogen, Groningen, The Netherlands) were cut with each 10 U of the restriction endonucleases XhoI and NotI (both Roche Diagnostics, Mannheim). 300 ng of the plasminogen expression plasmid pAC37.1 (see example 6a), were also cut with the enzymes XhoI and NotI. The thus treated DNA was separated by gel electrophoresis with a 0.9% agarose gel. The 2715 bp large plasminogen gene fragment from pAC37.1 as well as the 3073 bp large vector fragment from pGAPZaA were extracted from the gel by means of the QIAgen gel extraction kit (Qiagen, Hilden). The two fragments were combined and ligated at 4° C. over night with 1 U T4-DNA-ligase.

The transformation of E. coli DH5a, the isolation and the characterization of the resulting plasmid was carried out analogous to the description in example 1b, whereas instead of the antibiotic zeocin the antibiotic ampicillin was used for the selection of transformants. The thus constructed plasmid was referred to as pJW9.1 (FIG. 7).

### EXAMPLE 7b

### Transformation of Pichia pastoris with the Plasmid pJW9.1

As described for the transformation of Pichia pastoris KM71H with pMHS476.1 in example 1c, Pichia pastoris KM71H was transformed with the plasmid pJW9.1 linearized with the restriction endonuclease BlnI. The transformed cells were plated onto the YPDS-agar with 100 μg/ml zeocin (EasySelect™ Pichia Expression Kit instruction manual) and incubated. The obtained colonies were referred to as Pichia pastoris KM71H/pJW9.1-a, whereas “a” again represents the consecutive numbering of the colonies beginning at 1.

### EXAMPLE 7c

### Fermentation of Pichia pastoris KM71H/pJW9.1-3 for the Evaluation of the pH Value at the Glycerol Feed Rate

The precultures and the fermentation in the “stirrer-pro” were carried out as described in example 6i. The results are shown in tab. 2.

TAB 2

| Exp. | pH | substrate | feed rate | OD₆₀₀ | plasminogen conc. |
|---|---|---|---|---|---|
| I | 6.5 | glycerol | 1 ml/h | 220 | 18.6 mg/l |
| II | 7.0 | glycerol | 1 ml/h | 203 | 22.2 mg/l |
| III | 6.5 | glycerol | 0.5 ml/h | 142 | 10.1 mg/l |
| IV | 7.0 | glycerol | 0.5 ml/h | 99 | 3.8 mg/l |

Also in case of glycerol feed the best yields were obtained by fermentation at neutral pH value, whereas the influence of the substrate dosage (feed rate) on the product formation can be seen clearly.

### EXAMPLE 7d

### Fermentation of Pichia pastoris KM71H/pJW9.1-3

50 ml of YEP-G-medium (10 g/l yeast extract, 20 g/l casein peptone, 20 g/l glycerol) in a 1 l wide neck flask without baffles were inoculated with Pichia pastoris KM71H/pJW9.1-3 and incubated for 9 h at 30° C. and 300 rpm. 10 ml of this culture were used to inoculate 40 ml of MG-medium (5 g/l yeast nitrogen base w/o amino acids, 20 g/l glycerol, 2.5 ml/l biotin solution (0.2 g/l)) in a 1 l wide neck shaking flask without baffles. This culture was incubated for 16 h at 30° C. and 300 rpm.

3 l of BSM-medium (see example 6e) were inoculated with 30 ml of this culture in a 7.5 l laboratory fermenter (type Labfors, Infors AG, CH). The fermentation was carried out at 25° C. and a constant gas feed rate of 3.2 l/min. After 24 h glycerol solution (500g/l glycerol, 10 ml/l trace solution, 10 ml/l biotin solution [see example 6e]) was added. The dosage rate was increased step by step from 10 ml/h up to 45 ml/h during the fermentation. After 250 h a plasminogen activity of 1375 U/l could be measured after streptokinase activation.

### EXAMPLE 8

### Identification of Plasminogen Activators

24 commercially purchasable proteases were tested on their eligibility for the plasminogen activation. The experiments thereto were carried out in 100 mM sodium phosphate buffer pH 8, 0.36 mM CaCl₂, 0.9% NaCl.

The proteases supplied in a powdery form were dissolved in buffer, the proteases supplied in solution were used directly and diluted respectively with buffer if needed. 25 μl of the protease solutions were mixed with 25 μl plasminogen according to the present invention (20 mg/ml) and incubated for 10 min at 37° C. Afterwards the plasmin activity was measured with regard to the substrate N-tosyl-Gly-Pro-Lys-pNA. For this 200 μl substrate solution (9.5 mg N-tosyl-Gly-Pro-Lys-pNA, dissolved in 75 mg glycine/10 ml, 2% Tween® 20) were pipetted to 850 μl buffer, merged with the 50 μl of the preincubated plasminogen protease mixture and further incubated at 37° C. The increase of the extinction was measured photometrically at 405 nm. For the measurement of the extinction increase due to the proteases tests were carried out, in which instead of the preincubated plasminogen protease mixture a likewise preincubated buffer protease mixture was used.

The protease from S. griseus, protease Vil, protease XXIII, protease XIX, protease XVIII, ficin, metalloendopeptidase, clostripain, Glu-C, protease XIII, chymopapain, chymotrypsin, protease X, bromelain, kallikrein and proteinase A were purchased from Sigma, Deisenhofen; trypsin, papain, Asp-N, dispase 1, Lys-C, thrombin and elastase came from Roche, Mannheim; the proteinase K was supplied by QIAGEN, Hilden. The produced protease stock solutions had the protein concentrations given in the Table. 3. The dilution factor F indicates in which ratio the stock solutions are diluted for the measurements.

Following plasmin activities could be determined after activation (1 U/mg=1 μmol N-tosyl-Gly-Pro-Lys-pNA conversion per minute per mg protein):

TABLE 3

| | plasmin activity | conc. protein | |
|---|---|---|---|
| Protease | after activation | [mg/ml] | F |
| Protease from S. griseus | 613.3 U/mg | 0.77 | 1000 |
| Protease VIII | 9 U/mg | 3.58 | 1000 |
| Protease XXIII | * | 17.8 | 50000 |
| Protease XIX | * | 2.78 | 100 |
| Protease XVIII | 0.7 U/mg | 1.79 | 100 |
| Ficin | 0.01 U/mg | 0.81 | 1 |
| Metalloendopeptidase | 8.9 U/mg | 0.01 | 1 |
| Clostripain | 1.7 U/mg | 0.25 | 1 |
| Endopoteinase Glu-C | 0.6 U/mg | 0.81 | 1 |
| Protease XIII | 0.01 U/mg | 0.43 | 1 |
| Chymopapain | * | 2.02 | 1 |
| Chymotrypsin | * | 0.14 | 1 |
| Protease X | * | 2.01 | 1 |
| Bromelain | * | 0.81 | 1 |
| Kallikrein | * | 0.56 | 1 |
| Proteinase A | 0.02 U/mg | 0.36 | 1 |
| Trypsin | 11 kU/mg | 3.40 | 100000 |
| Papain | * | 0.64 | 10 |
| Endoproteinase Asp-N | 4.3 U/mg | 0.004 | 1 |
| Dispase I | * | 0.2 | 1 |
| Endoproteinase Lys-C | * | 0.01 | 1 |
| Thrombin | 83.0 U/mg | 0.59 | 500 |
| Elastase | 0.63 U/mg | 0.36 | 5 |
| Proteinase K | * | 3.60 | 100 |

* For the proteases protease XXIII, protease XIX, chymopapain, endoproteinase Lys-C, chymotrypsin, papain, dispase I, protease X, bromelain, kallikrein and proteinase K no plasminogen activation could be detected.

### EXAMPLE 9

### Pharmaceutical Formulations

The recombinant functional plasminogen used in the following examples was obtained by means of the inventive method of production. In this connexion the term “plasminogen” refers to recombinant micro-, mini-, Lys- or Glu-plasminogen and the term “plasmin” to plasmin, which was obtained by proteolytic cleavage of recombinant micro-, mini-, Lys- or Glu-plasminogen. The activation of micro-, mini-, Lys- or Glu-plasminogen can be obtained by use of the same plasminogen activators, especially plasminogen activating proteases as described above, but is not limited to these examples, whereas the ratio of units activator to units plasminogen (micro-, mini-, Lys- or Glu-plasminogen) is about 1: 1000.

The plasminogen can be activated proteolytically, i.e. by the proteases tissue plasminogen activator, urokinase or the proteases protease VIII or protease from S. griseus described in the patent as well as by complexation with streptokinase or staphylokinase.

### EXAMPLE 9a

### Pharmaceutical Formulations

Hydrogels

Base formulation for hydrogels (100 g)

| | |
|---|---|
| Plasminogen | 100 U |
| Plasminogen activator(s) | 0.1 U |
| Hydroxyethyl cellulose 10 000 | 3.5 g |
| optional conservation (sorbinic acid/ | |
| potassium sorbate 0.1-0.4%, PHB-ester 0.1%) | |
| purified water | ad 100.0 |

The hydroxyethyl cellulose resp. instead of it hypromellose resp. methyl cellulose can be used alternatively in an amount of 0.5-15.0 g.

Gel

| | | |
|---|---|---|
| Plasminogen | 1000 | U |
| Plasminogen activator(s) | 1 | U |
| Glycerol (85%) | 150.0 | g |
| Hydroxyethyl cellulose 10 000 | 32.5 | g |
| optional conservation (sorbinic acid/ | | |
| potassium sorbate 0.1-0.4%, PHB-ester 0.1%) | | |
| Ringer's solution without lactate | ad 1000.0 | g |

alternatively:

100 g contain:

| | |
|---|---|
| Plasminogen | 100 U |
| Plasminogen activator(s) | 0.1 U |
| Hydroxyethyl cellulose 30 000 | 2.5 g |
| Glycerol 85% | 10.0 g |
| optional conservation (sorbinic acid/ | |
| potassium sorbate 0.1-0.2%, PHB-ester 0.1%) | |
| purified water | ad 100.0 |

alternatively:

100 g gel contain:

| Plasminogen | 100 | U |
|---|---|---|
| Plasminogen activator(s) | 0.1 | U |
| Polyacrylic acids | 1 | g |
| Propylene glycol | 8 | g |
| Mid-chained triglyceride | 8 | g |
| Diethylamine (for adjusting pH) | q.s. | |
| optional conservation (sorbinic acid/ | | |
| potassium sorbate 0.1-0.2%, PHB-ester 0.1%) | | |
| 2-Propanol | 0-1 | g |
| Water | ad 100 | g |

Hydrophilic Ointment (Macrogol Ointment)

50 g contain

| | | |
|---|---|---|
| Plasminogen | 50 | U |
| Plasminogen activator(s) | 0.05 | U |
| Macrogol 400 | 30.0 | g |
| Macrogol 4000 | 10.0 | g |
| optional conservation (sorbinic acid/ | | |
| potassium sorbate 0.1-0.2%, PHB-ester 0.1%) | | |
| Purified water | ad 50.0 | g |

alternatively:
Water-free Macrogol Ointment

100g contain:

| | | |
|---|---|---|
| Plasminogen | 100 | U |
| Plasminogen activator(s) | 0.1 | U |
| Macrogol 300 | 50 | g |
| Macrogol 1500 | ad 100 | g |

alternatively:

| | | |
|---|---|---|
| Plasminogen | 100 | U |
| Plasminogen activator(s) | 0.1 | U |
| Cetylstearyl alcohol | 29 | g |
| Paraffin, viscous | 34 | g |
| Vaseline, white | 100 | g |

Hydrophobic Ointment

| | | |
|---|---|---|
| Plasminogen | 100 | U |
| Plasminogen activator(s) | 0.1 | U |
| Vaseline | 80.0 | g |
| Paraffin thin fluid | ad 100 | g |

Hydrophobic Paste

| | | |
|---|---|---|
| Plasminogen | 100 | U |
| Plasminogen activator(s) | 0.1 | U |
| Hypromellose 400 | 20 | g |
| Vaseline, white | ad 100 | g |

alternatively:

| | | |
|---|---|---|
| Plasminogen | 100 | U |
| Plasminogen activator(s) | 0.1 | U |
| Carbomer (e.g. carbopol 974p) | 15 | g |
| Paraffin, viscous | 40 | g |
| Vaseline, white | ad 100 | g |

Creme

| | | |
|---|---|---|
| Plasminogen | 100 | U |
| Plasminogen activator(s) | 0.1 | U |
| Mid-chained triglycerides | 20 | g |
| Emulgating cetylstearyl alcohol | 10 | g |
| Lanolin | 10 | g |
| Sorbitol | 10 | g |
| optional conservation (sorbinic acid/ | | |
| potassium sorbate 0.1-0.2%, PHB-ester 0.1%) | | |
| purified water | ad 100 | g |

Nonionic Hydrophilic Creme

| | | |
|---|---|---|
| Plasminogen | 100 | U |
| Plasminogen activator(s) | 0.1 | U |
| Cetyl alcohol | 20 | g |
| 2-Ethyllauromyristat | 10 | g |
| Glycerol 85% | 6 | g |
| Potassium sorbate | 0.14 | g |
| Citric acid | 0.07 | g |
| Water | ad 100 | g |

Nonionic Creme

| | | |
|---|---|---|
| Plasminogen | 100 | U |
| Plasminogen activator(s) | 0.1 | U |
| Polysorbat 60 | 5 | g |
| Cetylstearyl alcohol | 10 | g |
| Glycerol 85% | 10 | g |
| Vaselin, white | 25 | g |
| optional conservation (sorbinic acid/ | | |
| potassium sorbate 0.1-0.2%, PHB-ester 0.1%) | | |
| Water | ad 100 | g |

Liposomal Formulation

| | | |
|---|---|---|
| Plasminogen | 100 | U |
| Plasminogen activator(s) | 0.1 | U |
| Soja lecithin, Chicken lecithin | 15 | g |
| optional conservation (sorbinic acid/ | | |
| potassium sorbate 0.1-0.2%, PHB-ester 0.1%, | | |
| resp. diazodinyl urea 1-2 g) | | |
| Water | ad 100.0 | g |

Capsule

One capsule with 0.25g powder/granulate contains:

| | | |
|---|---|---|
| Plasminogen | 5 | U |
| Plasminogen activator(s) | 0.005 | U |
| Starch | 0.1 | g |
| Siliciumdioxide | 0.02 | g |
| Magnesium stearate | 0.002 | g |
| Polymethacrylate copolymerisates/polymethacrylic acid | 0.015 | g |
| Triethylcitrate | 0.0005 | g |
| Talkum | 0.001 | g |
| Cellulose, microcrystalline | ad 0.25 | g |

alternatively:

One capsule with 0.25 g powder/granulate contains:

| | | |
|---|---|---|
| Plasminogen | 5 | U |
| Plasminogen activator(s) | 0.005 | U |
| Siliciumdioxide | 0.01 | g |
| Magnesiumstearat | 0.002 | g |
| Polymethacrylat copolymerisates/ | 0.015 | g |
| polymethacrylic acid | | |
| Triethylcitrate | 0.0001 | g |
| Talkum | 0.001 | mg |
| Mannitol | ad 0.25 | g |

Pill

100 mg pill granulate contain:

| | | |
|---|---|---|
| Plasminogen | 5 | U |
| Plasminogen activator(s) | 0.005 | U |
| Starch | 30 | mg |
| Siliciumdioxide | 2 | mg |
| Magnesiumstearate | 4 | mg |
| Polymethacrylate copolymerisates/polymethacrylic acid | 5 | mg |
| Triethylcitrate | 0-1 | mg |
| Talkum | 0.0001 | mg |
| Cellulose, microcrystalline | ad 100 | mg |

Pellets

100 g pellets contain:

| | | |
|---|---|---|
| Plasminogen | 2000 | U |
| Plasminogen activator(s) | 2 | U |
| Starch | 20 | g |
| Sucrosestearate | 20 | g |
| Siliciumdioxide | 2 | g |
| Magnesiumstearate | 3 | g |
| Polyvinylpyrrolidone | 0-1 | g |
| Polymethacrylate copolymerisates/polymethacrylic acid | 5 | g |
| Talkum | 0.2 | g |
| Triethylcitrate | 0.1 | g |
| Cellulose, microcrystalline | ad 100 | g |

Injection Solution

| Plasminogen | 500 | U |
|---|---|---|
| Plasminogen activator(s) | 0.5 | U |
| Ethanol | 0-1 | g |
| Propylene glycol | 10 | g |
| Polyethylene glycol | 0-1 | g |
| Sodium chloride | q.s. | |
| optional buffer (sodium hydrogen phosphate/ | | |
| sodium dihydrogen phosphate) | | |
| purified Water | ad 100 | ml |

Instead of micro-, mini-, Lys- or Glu-plasminogen in case of the numerated formulations also the same amount based on the activity of plasmin can be used. If plasmin is used directly, no plasminogen activator(s) has/have to be contained in the pharmaceutical formulation.

### EXAMPLE 9b

### Pharmaceutical Formulations

a) Hydrogels

Basic formulation for hydrogels (100 g)

| | | |
|---|---|---|
| Plasmin | 100 | U |
| Hydroxyethyl cellulose 400 | 2.5-5.0 | g |
| purified water | ad 100.0 | g |

The time for swelling takes 1 to 3 h.

The use of 1-1000 U plasmin per gramme hydrogel is possible.

b) Hydrophilic Ointment

Basic formulation of a hydrophilic ointment (1000 g):

| | | |
|---|---|---|
| Plasmin | 1000 | U |
| Glycerol, water-free | 85.0 | g |
| Hydroxyethyl cellulose 10.000 | 32.5 | g |
| optionally polyhexanide | 0.2 | weight- % |
| Ringer's solution without lactate | ad 1000.0 | g |

Polyhexanide can be added optionally as antimicrobial active agent in a concentration up to 0.2 weight-%. Instead of hydroxyethyl cellulose 10.000 (Natrosol 250© HX PHARM) also hydroxyethyl cellulose 400 (e.g. Tylose© H 300 or Natrosol 250© HX PHARM) can be added.

The use of 1-10000 U plasmin per gramme ointment is possible.

c) Ointment

Basic formulation for ointment (50 g)

| | | |
|---|---|---|
| Plasmin | 50 | U |
| Macrogol 400 | 30.0-32.5 | g |
| Macrogol 4000 | 12.5-7.5 | g |
| purified water | ad 50.0 | g |

Preparation:

12.5 g macrogol 4000 and 30.0 g macrogol 400 (in case of supple ointments 7.5 g macrogol 4000 and 32.5 g macrogol 400) are heated in the water bath in an ointment dish until the smelting of the macrogol. After cooling down the appropriate amount of plasmin, which was produced by means of the inventive method, dissolved in 7.5 g of purified water is added and afterwards homogenized.

d) Capsule

Basic formulation for 0.5 g

| | | |
|---|---|---|
| Plasmin | 5 | U |
| Lactose | 0.42 | g |
| Starch | 0.06 | g |
| Magnesium stearate | 0.02 | g |

The use of 0.1-100 U plasmin per capsule is possible.

e) Injection Solution/Infusion Solution

Basic formulation for 100 ml

| | | |
|---|---|---|
| Plasmin | 500 | U |
| Ethanol | 0.01 | g |
| Propylene glycol | 30 | ml |
| purified water | ad 100 | ml |

The use of 1-500 U plasmin per ml of solution is possible.

Instead of plasmin also micro-, mini-, Lys- or Glu-plasminogen can be used in the mentioned amounts for the plasmin based on the activity in units, if at the same time at least one plasminogen activator is added in an amount of 1:10000 to 1:100, preferred in an amount of 1: 1000 based on the plasminogen activity.

### EXAMPLE 10a

### Amplification and Cloning of Different Forms of the Mini- and the Micro-Plasminogen Gene and Cloning into the Vector pPICZαA; Transformation of Pichia pastoris

Mini- and micro-plasminogen represent shortened plasminogen derivatives, which are lacking of the N-terminal domains, but which are still activable into active plasmin. The amplification of the mini- and micro-plasminogen genes for cloning into the vector pPICZaA was carried out with the oligonucleotide primer N034 for the 3′-end and in each case with one of the primers N036a-j (Seq. ID No. 19 to 28) for the particular 5′-end in using the conditions described in example 1a. The oligonucleotide primers N036a,c,e,g,i have beside the bases complementary to the plasminogen gene the codons for the Kex2 cleavage site, the primers N036b,d,f,h,j have in addition subsequent to the codons for the Kex2 cleavage site the codons for two Ste13 cleavage sites. The primer N034 has further on a Kspl cleavage site, the primers N036 a-j have a XhoI cleavage site.

The cloning of the mini- and micro-plasminogen genes into the vector pPICZaA was carried out analogous to the cloning described in example 1b, whereas the vector as well as the particular PCR product were cut with the restriction endonucleases XhoI and KspI. The used primers, the names of the plasminogen derivative, the coded protease cleavage sites, the labeling of the obtained plasmids and the N-terminal amino acid of the secreted plasminogen derivative are summarized in the following table.

| | | | protease- | | N-terminal |
|---|---|---|---|---|---|
| 5′-primer | 3′-primer | name | cleavage site | plasmid name | amino acid* |
| N036a | N034 | mini-plasminogen | Kex2 | pPLG1.1 | A463 |
| N036b | N034 | mini-plasminogen | Kex2, 2xSte13 | pPLG2.1 | A463 |
| N036c | N034 | micro-plasminogen | Kex2 | pPLG3.2 | K550 |
| N036d | N034 | micro-plasminogen | Kex2, 2xSte13 | pPLG4.2 | K550 |
| N036e | N034 | micro-plasminogen | Kex2 | pPLG5.3 | L551 |
| N036f | N034 | micro-plasminogen | Kex2, 2xSte13 | pPLG6.1 | L551 |
| N036g | N034 | micro-plasminogen | Kex2 | pPLG7.1 | A562 |
| N036h | N034 | micro-plasminogen | Kex2, 2xSte13 | pPLG8.3 | A562 |
| N036i | N034 | micro-plasminogen | Kex2 | pPLG9.1 | S564 |
| N036j | N034 | micro-plasminogen | Kex2, 2xSte13 | pPLG10.1 | S564 |

*The numeration refers to the 810 amino acid long preplasminogen (Seq. ID No. 12)

FIG. 8 shows exemplary the plasmid pPLG1.1.

As described for pMHS476.1 in example 1c Pichia pastoris KM71H was transformed with the plasmid pPLG1.1. The obtained colonies were referred to as Pichia pastoris KM71H/pPLG1.1-1/a, whereas “a” again represents the consecutive numbering of the colonies beginning at 1.

The generation of strains on basis of the plasmids pPLG2.1, pPLG3.2, pPLG4.2, pPLG5.3, pPLG6.1, pPLG7.1, pPLG8.3, pPLG9.1 and pPLG10.1 was carried out according to the production of the strain KM71H/pPLG1.1-1/a.

Oligonucleotide Primer N036a-j

N036a
AAAAACTCGAGAAAAGAGCACCTCCGCCTGTTG
N036b
AAAAACTCGAGAAAAGAGAGGCTGAAGCTGCACCTCCGCCTGTTG
N036c
AAAAACTCGAGAAAAGAAAACTTTACGACTACTG
N036d
AAAAACTCGAGAAAAGAGAGGCTGAAGCTAAACTTTACGACTACTG
N036e
AAAAACTCGAGAAAAGACTTTACGACTACTGTG
N036f
AAAAACTCGAGAAAAGAGAGGCTGAAGCTCTTTACGACTACTGTG
N036g
AAAAACTCGAGAAAAGAGCCCCTTCATTTGATTGTG
N036h
AAAAACTCGAGAAAAGAGAGGCTGAAGCTGCCCCTTCATTTGATTGTG
N036i
AAAAACTCGAGAAAAGATCATTTGATTGTGGGAAGCC
N036j
AAAAACTCGAGAAAAGAGAGGCTGAAGCTTCATTTGATTGTGGGAAGCC

### EXAMPLE 10b

### Amplification and Cloning of Different Forms of the Mini- and the Micro-Plasminogen Gene and Cloning into the Vector pGAPZαA; Transformation of Pichia pastoris

The amplification of the mini- and micro-plasminogen genes for cloning into the vector pGAPZaA was carried out with the oligonucleotide primer N034 for the 3′-end and in each case with one of the primers N036a-j (Seq. ID No. 19 to 28) for the particular 5′-end in using the conditions described in example 1a. The oligonucleotide primers N036a,c,e,g,i have beside the bases complementary to the plasminogen gene the codons for the Kex2 cleavage site, the primers N036b,d,f,h,j have in addition subsequent to the codons for the Kex2 cleavage site the codons for two Ste13 cleavage sites. The primer N034 has further on a Kspl cleavage site, the primers N036 a-j have a XhoI cleavage site.

The cloning of the mini- and micro-plasminogen genes into the vector pGAPZaA was carried out analogous to the cloning described in example 1b, whereas the vector as well as the particular PCR product were cut with the restriction endonucleases XhoI and KspI. Summarized the used primers, the names of the plasminogen derivative, the coded protease cleavage sites, the labeling of the obtained plasmids and the N-terminal amino acid of the secreted plasminogen derivative can be taken from the following table.

| | | | protease- | | N-terminal |
|---|---|---|---|---|---|
| 5′-primer | 3′-primer | name | cleavage site | plasmid name | amino acid* |
| N036a | N034 | mini-plasminogen | Kex2 | pPLG11.2 | A463 |
| N036b | N034 | mini-plasminogen | Kex2, 2xSte13 | pPLG12.1 | A463 |
| N036c | N034 | micro-plasminogen | Kex2 | pPLG13.1 | K550 |
| N036d | N034 | micro-plasminogen | Kex2, 2xSte13 | pPLG14.2 | K550 |
| N036e | N034 | micro-plasminogen | Kex2 | pPLG15.1 | L551 |
| N036f | N034 | micro-plasminogen | Kex2, 2xSte13 | pPLG16.3 | L551 |
| N036g | N034 | micro-plasminogen | Kex2 | pPLG17.2 | A562 |
| N036h | N034 | micro-plasminogen | Kex2, 2xSte13 | pPLG18.1 | A562 |
| N036i | N034 | micro-plasminogen | Kex2 | pPLG19.2 | S564 |
| N036j | N034 | micro-plasminogen | Kex2, 2xSte13 | pPLG20.1 | S564 |

*The numeration refers to the 810 amino acid long preplasminogen (Seq. ID No. 12)

FIG. 9 shows exemplary the plasmid pPLG11.2.

As described for pJW9.1 in example 7a Pichia pastoris KM71H was transformed with the plasmid pPLG11.2 linearized by the restriction endonuclease BlnI. The obtained colonies were referred to as Pichia pastoris KM71H/pPLG11.2-1/a, whereas “a” again represents the consecutive numbering of the colonies beginning at 1.

The generation of strains on basis of the plasmids pPLG12.1, pPLG13.1, pPLG14.2, pPLG15.1, pPLG16.3, pPLG17.2, pPLG18.1, pPLG19.2 and pPLG20.1 was carried out according to the production of the strain KM71H/pPLG1.1-1/a.

### Bibliography

(1)=Desire Collen, Thrombosis and Haemostasis, 82, 1999
(2)=Forsgren et al., FEBS Lett. 213, 1987
(3)=Petersen et al., J. Biol. Chem., 265, 1990
(4)=Duman et al., Biotechnol. Appl. Biochem. 28; 39-45, 1998
(5)=Guan et al., Sheng Wu Gong Cheng Xue Bao, 17, 2001
(6)=Gonzalez-Gronow et al., Biochimica et Biophysica Acta, 1039, 1990
(7)=Whitefleet-Smith et al., Arch. Biochem. Biophys., 271, 1989
(8)=Nilsen und Castellino, Protein Expression and Purification, 16, 1999
(9)=Busby et al., J. Biol. Chem., 266, 1991
(10)=Maniatis et al., Molecular Cloning: A Laboratory Manual, Cold Spring Harbor press, 1989
(11)=Gassen & Schrimpf, Gentechnische Methoden, Spektrum Akademischer Verlag, Heidelberg, 1999
(12)=Malinowski et al., Biochemistry, 23, 1984
(13)=Stack et al., Biochem. J. 284, 1992

1. A method for recombinant production of a functional plasminogen in microorganisms comprising at least the step:
a) fusion of a nucleic acid sequence coding for at least the functional part of the plasminogen with a nucleic acid sequence coding for at least one signal peptide, the nucleic acid sequence coding for the functional plasminogen and the nucleic acid sequence coding for at least the signal peptide being coupled with codons for cleavage sites of proteases providing for the cleavage of the signal peptide.

2. Method for recombinant production according to claim 1, wherein the nucleic acid sequence coding for at least the functional part of the plasminogen comprises the proteolytic domain of plasminogen or a mutant or a fragment thereof.

3. Method for recombinant production according to claim 1, wherein the nucleic acid sequence coding for at least the functional part of the plasminogen codes for Glu- or Lys-plasminogen.

4. Method for recombinant production according to claim 1, wherein the nucleic acid sequence coding for at least one signal peptide codes for a prepropeptide, a prepeptide or a propeptide and/or wherein the codons code for cleavage sites of proteases for the protease Kex2 and/or Ste13.

5. Method for recombinant production according to claim 4, characterized in that the nucleic acid molecule coding for at least one signal peptide codes for the signal peptide of the alpha-factor or of SUC2, PHA-E or PHOL from the yeast Saccharomyces cerevisiae.

6. Method for recombinant production according to claim 1, characterized in that at least 20 mg/l of functional Glu- or Lys-plasminogen are produced.

7. Method for recombinant production according to claim 1, characterized in that after a processing of 120 hours at least 120 U/l of functional Lys-plasminogen are produced.

8. Method for recombinant production according to claim 1, wherein as primers for the amplification are used two oligonucleotide primers selected from the group comprising N036a (Seq. ID No. 19), N036b (Seq. ID No. 20), N036c (Seq. ID No. 21), N036d (Seq. ID No. 22), N036e (Seq. ID No. 23), N036f (Seq. ID No. 24), N036g (Seq. ID No. 25), N036h (Seq. ID No. 26), N036i (Seq. ID No. 27), and N036j (Seq. ID No. 28).

9. Method for recombinant production according to claim 1, wherein as primers for the amplification are used two oligonucleotide primers selected from the group comprising N034 (Seq. ID No. 1), N036 (Seq. ID No. 2), N057 (Seq. ID No. 3), N037 (Seq. ID No. 4), N035 (Seq. ID No. 5), N056 (Seq. ID No. 6), as well as from the group comprising N036a (Seq. ID No. 19), N036b (Seq. ID No. 20), N036c (Seq. ID No. 21), N036d (Seq. ID No. 22), N036e (Seq. ID No. 23), N036f (Seq. ID No. 24), N036g (Seq. ID No. 25), N036h (Seq. ID No. 26), N036i (Seq. ID No. 27), and N036j (Seq. ID No. 28).

10. Method for recombinant production according to claim 1, wherein the fusion product resulted in step a) is incorporated into an expression vector suitable for microorganisms.

11. Method for recombinant production according to claim 10, wherein the expression vector being suitable for fungi.

12. Method for recombinant production according to claim 10, wherein the expression vector comprises an inducible or constitutive promoter.

13. Method for recombinant production according to claim 10, wherein the expression vector comprises the constitutive GAP-promoter from P. pastoris.

14. Method for recombinant production according to claim 12, characterized in that the thus obtained nucleic acid is a plasmid preferably selected from the group pPLG11.2, pPLG12.1, pPLG13.1, pPLG14.2, pPLG15.1, pPLG16.3, pPLG17.2, pPLG18.1, pPLG19.2, pPLG20.1, pAC37.1, pJW9.1, pMHS476.1, pSM54.2, pSM49.8, pSM82.1, and pSM58.1.

15. Method for recombinant production according to claim 1, characterized in that the codons code for the cleavage site of the protease Kex2 and the fusion gene of the plasminogen has the nucleic acid sequence shown in Seq. ID No. 7, 13, 50, 52, 54, 56 or 58.

16. Method for recombinant production according to claim 1, characterized in that the codons code for the cleavage site of the protease Kex2 and the fusion protein of the plasminogen has the amino acid sequence shown in Seq. ID No. 8, 14, 40, 42, 44, 46 or 48.

17. Method for recombinant production according to claim 1, characterized in that the codons code for the cleavage site of the protease Kex2 and the protease Ste13 and the fusion gene of the plasminogen has the nucleic acid sequence shown in Seq. ID No. 9, 15, 51, 53, 55 or 57.

18. Method for recombinant production according to claim 1, characterized in that the codons code for the cleavage site of the protease Kex2 and the protease Ste13 and the fusion protein of the plasminogen has the amino acid sequence shown in Seq. ID No. 10, 16, 41, 43, 45, 47 or 49.

19. Method for recombinant production according to claim 1, wherein a host accounted to the microorganisms is transformed with the plasmid included in claim 14.

20. Method for recombinant production according to claim 1, wherein the used host organism is a eucaryotic microorganism.

21. Method for recombinant production according to claim 20, characterized in that the used host organism is accounted to the genus of fungi.

22. Method for recombinant production according to claim 21, characterized in that the used host organism is accounted to the genus Pichia, Saccharomyces, Hansenula, Candida or Aspergillus.

23. Method for recombinant production according to claim 22, characterized in that the used host organism is accounted to the species Pichia pastoris, Pichia methanolica, Hansenula polymorpha, Aspergillus niger, Aspergillus oryzae or Aspergillus nidulans.

24. Method for recombinant production according to claim 1, wherein the nucleic acid sequence being over-expressed coding for at least the functional part of the plasminogen and at least the functional part of plasminogen being secreted from a microbial host organism transformed with the fusion product generated according to step a).

25. Method for recombinant production according to claim 1, wherein the functional part of the nucleic acid sequence of plasminogen being one of the sequences Seq. ID No. 60, 61, 62, 63, 64, 65 or 66.

26. Method for recombinant production according to claim 1, characterized in that a functional human plasminogen is produced.

27. Method for recombinant production according to claim 1, characterized in that a functional human-homologous plasminogen is produced.

28. Plasmid pPLG11.2, plasmid pPLG12.1, plasmid PPLG13.1, plasmid pPLG14.2, plasmid pPLG15.1, plasmid pPLG16.3, plasmid pPLG17.2, plasmid pPLG18.1, plasmid pPLG19.2, or plasmid pPLG20.1.

29. Plasmid pMHS476.1, plasmid pSM54.2, plasmid pSM49.8, plasmid pSM82.1.

30. Plasmid pSM58.1, plasmid pAC37.1, plasmid pJW9.1.

31. Nucleic acid sequence obtainable via the method of recombinant production according to claim 1, characterized in that the nucleic acid sequence coding for at least the functional part of plasminogen is operatively coupled to a promoter being active in the yeast and further on coupled to a nucleic acid sequence coding for at least one signal peptide, the nucleic acid sequence coding for the functional plasminogen and the nucleic acid sequence coding for at least the signal peptide being coupled with codons for the cleavage sites of the proteases Kex2 and Ste13.

32. Plasminogen obtainable via the method of recombinant production according to claim 1.

33. Plasminogen according to claim 32, characterized in that a microplasminogen, miniplasminogen, Lys-plasminogen, Glu-plasminogen or a plasminogen derivative is concerned.

34. Plasminogen derivative according to claim 33, characterized in that it contains the functional proteolytic domain of the plasminogen and comprises at least one deletion and/or at least one amino acid exchange and/or is fused with at least one other amino acid or at least one peptide or at least one protein.

35. Plasminogen derivative according to claim 33, characterized in that it is activable via at least one plasminogen activator to active plasmin.

36. Plasminogen derivative according to claim 33, characterized in that it contains at least the functional proteolytic domain of plasminogen and features a sequence homology to micro-, mini-, Lys- or Glu-plasminogen of above 80%, preferred of above 90% and especially preferred of above 95%.

37. Plasmin obtainable via activation of plasminogen or a plasminogen derivative according to claim 32 using at least one plasminogen activator.

38. Microbial host organism comprising the fusion product or one nucleic acid sequence derived thereof obtained in step a) according to claim 1.

39. Microbial host organism according to claim 38, characterized in that it is selected from the group Pichia pastoris, Pichia methanolica, Saccharomyces cerevisiae, Hansenula polymorpha, Aspergillus niger, Aspergillus oryzae and Aspergillus nidulans.

40. Use of a functional plasminogen and/or the plasmin emerging thereof produced according to claim 1 for the preparation of a pharmaceutical.

41. Use of a functional plasminogen and/or the plasmin emerging thereof produced according to claim 1 for the treatment of wounds, thrombotic events or for the prevention of thrombotic events.

42. Use of a functional plasminogen and/or the plasmin emerging thereof produced according to claim 1 as anti-thrombotic as well as anti-coagulative active agents.

43. Use more particularly according to claim 42 for prophylaxis and/or treatment of heart attack, apoplexy, thrombosis, venous thromboses, restenosis, hypoxia, ischemia, coagulation necrosis, inflammations of the blood vessels, acute lung embolia, acute and subacute arterial thromboses, fresh or older coagulations of venous thromboses, deep venous thromboses of the hip and the extremities, early thromboses in the area of desobliterated vessels, acute central vessel occlusion at the eye, conjunctivitis in case of plasminogen type-I deficiency, burn injuries, alkali or acid burns and frostbites, disseminated intravasal coagulation during shock, acute arterial occlusions of the extremities, chronic occlusive arteriopathies, thrombosis of arteriovenous shunts as well as for treatment subsequent to a heart attack, subsequent to a bypass surgery, subsequent to an angioplasty as well as subsequent to a balloon dilatation, for the thrombolytic therapy in the case of acute heart attack, for recanalization of arteriovenous shunts as well as for the reperfusion of occluded coronary arteries in the case of acute heart attack.

44. Use according to claim 40 in combination with an anticoagulant.

45. Use according to claim 44, characterized in that the anticoagulant concerns heparin, heparin derivatives or acetylsalicylic acid.

46. Use of a functional plasminogen and/or the plasmin emerging thereof produced according to claim 1 for incorporating into dressing materials, plasters or for the use in combination with vulnerary drugs.

47. Dressing materials, vulnerary bandages and plasters comprising functional plasminogen and/or the plasmin emerging thereof produced according to claim 1.

48. Dressing materials, vulnerary bandages and plasters according to claim 47, comprising 0.01-500 U plasminogen and/or a plasmin emerging thereof per cm2 of pharmaceutical formulation, preferred 0.1-250 Units and especially preferred 1-150 Units plasminogen and/or a plasmin emerging thereof per cm2 pharmaceutical formulation.

49. Use of the dressing materials, vulnerary bandages and plasters according to claim 47 for treatment of burn injuries, frostbites, alkali or acid burns, injuries and/or wounds.

50. Pharmaceutical composition comprising a plasminogen and/or a plasmin emerging thereof produced according to claim 1, and a pharmaceutically acceptable carrier, additive and/or solvent as well as in case of need anticoagulative active agents.

51. Pharmaceutical composition according to claim 50 suitable for oral, topical or parenteral, intravasal, especially intravenous, intraperitoneal, subcutaneous or intramuscular application.

52. Pharmaceutical composition according to claim 51 suitable for topical application containing 0.01-500 U plasminogen and/or a plasmin emerging thereof per gram of the pharmaceutical composition preferred 0.1-250 Units and especially preferred 1-150 Units plasminogen and/or a plasmin emerging thereof per gram of the pharmaceutical composition.

53. Pharmaceutical composition according to claim 51 suitable for oral application containing 0.1-100.000 U plasminogen and/or a plasmin emerging thereof per gram of the pharmaceutical composition preferred 100-80.000 Units and especially preferred 1.000-50.000 Units plasminogen and/or a plasmin emerging thereof per gram of the pharmaceutical composition.

54. Pharmaceutical composition according to claim 51 suitable for injection or infusion containing 0.1-100 million U plasminogen and/or a plasmin emerging thereof per 10 ml solution preferred 1-10 million Units and especially preferred 3-5 million Units plasminogen and/or a plasmin emerging thereof per 10 ml of the injection or infusion solution.

55. Use of the pharmaceutical composition according to claim 50 for prophylaxis and/or treatment of heart attack, apoplexy, thrombosis, venous thromboses, restenosis, hypoxia, ischemia, coagulation necrosis, inflammations of the blood vessels, acute lung embolia, acute and subacute arterial thromboses, fresh or older coagulations of venous thromboses, deep venous thromboses of the hip and the extremities, early thromboses in the area of desobliterated vessels, acute central vessel occlusion at the eye, conjunctivitis in case of plasminogen type-I deficiency, burn injuries, alkali or acid burns and frostbites, disseminated intravasal coagulation during shock, acute arterial occlusions of the extremities, chronic occlusive arteriopathies, thrombosis of arteriovenous shunts as well as for treatment subsequent to a heart attack, subsequent to a bypass surgery, subsequent to an angioplasty as well as subsequent to a balloon dilatation, for the thrombolytic therapy in the case of acute heart attack, for recanalization of arteriovenous shunts as well as for the reperfusion of occluded coronary arteries in the case of acute heart attack.

56. Vector comprising the fusion product or a nucleic acid sequence derived thereof obtained in step a) according to claim 1.

57. DNA-molecule comprising the fusion product or a nucleic acid sequence derived thereof obtained in step a) according to claim 1.

58. RNA-molecule comprising the fusion product or a nucleic acid sequence derived thereof obtained in step a) according to claim 1.

59. Screening method for the identification of plasminogen activators via use of the functional plasminogen according to claim 36.

60. Screening method for the identification of plasminoqen activators via use of the functional plasminogen, characterized in that the resulting plasmin activity is measured subsequent to preincubation of the proteases with the functional plasminogen according to claim 35.

61. Screening method according to claim 59, characterized in that the resulting plasmin activity is measured with a synthetic peptide substrate.

62. Screening method according to claim 61, characterized in that the resulting plasmin activity is measured with N-tosyl-Gly-Pro-Lys-pNA.
